(19) United States
(12) Patent Application Publication
(10) Pub. No.: US 2023/0191007 A1
(43) Pub. Date: Jun. 22, 2023

(54) BLOOD BAG SYSTEM AND CLAMP

(71) Applicant: TERUMO KABUSHIKI KAISHA, Tokyo (JP)

(72) Inventors: Yuusuke YAMAZAKI, Tokyo (JP); Masatsugu IGARASHI, Kanagawa (JP); Mayu KATAGIRI, Shizuoka (JP)

(21) Appl. No.: 18/170,436

(22) Filed: Feb. 16, 2023

Related U.S. Application Data
(63) Continuation of application No. PCT/JP2021/029888, filed on Aug. 16, 2021.

(30) Foreign Application Priority Data
Aug. 18, 2020 (JP) 2020-137885

(51) Int. Cl. A61M 1/02; A61M 1/36; A61M 39/28

(52) U.S. Cl. CPC A61M 1/0218; A61M 1/3693; A61M 39/28

(57) ABSTRACT

A blood bag system includes one clamp mounted over a second tube and a third tube. In a first insertion hole of a main body of the clamp, a portion of a first blocking portion excluding a portion connected with a first opening portion is closed by the main body. The main body can be deformed from a first state to a second state. In the first state, the third tube having been moved into a second blocking portion of a second insertion hole is restrained by the second blocking portion. In the second state, the third tube can be moved from the second blocking portion to a position that is other than a second opening portion and in which a third flow path of the third tube is reopened.

### CROSS-REFERENCE TO RELATED APPLICATIONS

The present disclosure is a continuation of and claims benefit to PCT/JP2021/029888 filed on Aug. 16, 2021, entitled “BLOOD BAG SYSTEM AND CLAMP” which claims priority to Japanese Patent Application No. 2020-137885A filed on Aug. 18, 2020. The entire disclosures of the applications listed above are hereby incorporated herein by reference, in their entireties, for all that they teach and for all purposes.

### BACKGROUND

The present disclosure relates to a blood bag system having a plurality of tubes and a clamp for opening and closing flow paths of the tubes, as well as the clamp.

In Japanese Patent Application No. 2016-106012 A, a blood bag system is disclosed, that is equipped with a first bag (a parent bag) for accommodating blood, a second bag (a child bag) for accommodating blood components obtained by centrifugally separating the blood inside the first bag, and a third bag (a chemical solution bag) accommodating an additive solution. The blood bag system includes a first tube connected with the first bag, a second tube connected with the second bag, and a third tube connected with the third bag, and the first to third tubes are connected with each other by way of a branching portion (a Y-type connector).

By being set in a centrifugal separation and transferring device by a user such as a medical worker or the like, the blood bag system constitutes a centrifugal separation system in which the blood is subjected to centrifugal separation to generate blood components, and a liquid containing the blood components is transferred between the first to third bags. In the centrifugal separation system, after a blood component has been transferred from the first bag to the second bag, a flow path of the second tube is blocked by the user by a first clamp, whereas a flow path of the third tube is blocked by a second clamp. In this state, the user takes out the blood bag system from the centrifugal separation and transferring device, suspends the blood bag system on a suspension platform, and further separates the second clamp from the third tube, thereby transferring the additive solution from the third bag into the first bag.

### BRIEF SUMMARY

Incidentally, in such a blood bag system, with the first clamp and the second clamp being provided as separate components, it is likely that the user will consume time and effort in order to respectively block the flow path of the second tube and the flow path of the third tube. Further, in an operation of unfastening the second clamp, there is a concern that the user may accidentally unfasten the first clamp. In other words, there is a concern that the wrong tube may be opened by mistake.

The present disclosure has been devised taking into consideration the aforementioned problems, and has at least one object of providing a blood bag system and a clamp, that are capable of efficiently blocking the flow paths of two tubes by a single clamp, and preventing the flow path of the tube from being opened by mistake.

A first aspect of the present disclosure is characterized by a blood bag system, comprising a first bag configured to accommodate blood, a second bag configured to accommodate a blood component obtained by subjecting the blood inside the first bag to centrifugal separation, a third bag configured to accommodate an additive solution, a first tube connected to the first bag, a second tube connected to the second bag, and a third tube connected to the third bag, the first tube, the second tube, and the third tube being connected via a branching portion, the blood bag system further comprising a single clamp mounted over the second tube and the third tube, wherein the clamp includes a main body, a first insertion hole that penetrates, or passes, through the main body and in which the second tube is arranged, and a second insertion hole that penetrates, or passes, through the main body and in which the third tube is arranged, the first insertion hole includes a first opening portion configured to place a flow path of the second tube in an open state, and a first blocking portion connected with the first opening portion and configured to place the flow path of the second tube in a blocked state, and in the first blocking portion, a portion thereof except for a portion connected with the first opening portion is closed by the main body, the second insertion hole includes a second opening portion configured to place a flow path of the third tube in an open state, and a second blocking portion connected with the second opening portion and configured to place the flow path of the third tube in a blocked state, the main body is configured to be changed from a first state to a second state, in the first state, the third tube, that has been moved into the second blocking portion from the second opening portion, is restrained by the second blocking portion, and in the second state, the third tube is allowed to be moved from the second blocking portion to a position that is a position other than the second opening portion and in which the flow path of the third tube is reopened.

Furthermore, in order to realize the aforementioned objects, a second aspect of the present disclosure is characterized by a clamp mounted over two tubes, the clamp comprising a main body, a first insertion hole that penetrates, or passes, through the main body and in which one tube of the two tubes is arranged, and a second insertion hole that penetrates, or passes, through the main body and in which another tube of the two tubes is arranged, wherein the first insertion hole includes a first opening portion configured to place a flow path of the one tube in an open state, and a first blocking portion connected with the first opening portion and configured to place the flow path of the one tube in a blocked state, and in the first blocking portion, a portion thereof except for a portion connected with the first opening portion is closed by the main body, the second insertion hole includes a second opening portion configured to place a flow path of the another tube in an open state, and a second blocking portion connected with the second opening portion and configured to place the flow path of the another tube in a blocked state, the main body is configured to be changed from a first state to a second state, in the first state, the another tube, that has been moved into the second blocking portion from the second opening portion, is restrained by the second blocking portion, and in the second state, the another tube is allowed to be moved from the second blocking portion to a position that is a position other than the second opening portion and in which the flow path of the another tube is reopened.

With the blood bag system and the clamp described above, it is possible to efficiently block the flow paths of the two tubes by a single clamp, and prevent the flow path of the tube from being opened by mistake.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages are described herein and will be apparent to those skilled in the art upon consideration of the following Detailed Description and in view of the figures.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is an explanatory diagram showing an overall configuration of a blood bag system and a centrifugal separation system according to a first embodiment of the present disclosure;

FIG. 2 is a plan view showing a unit setting area of a centrifugal separation and transferring device in accordance with embodiments of the present disclosure;

FIG. 3 is a perspective view showing a state in which a clamp of the blood bag system is set in a holder of the centrifugal separation and transferring device in accordance with embodiments of the present disclosure;

FIG. 4A is a perspective view showing a first state of the clamp in accordance with embodiments of the present disclosure;

FIG. 4B is a perspective view showing a second state of the clamp shown in FIG. 4A;

FIG. 5A is a cross-sectional view taken along line VA-VA of FIG. 4A;

FIG. 5B is an enlarged front view showing a piece portion of the clamp in accordance with embodiments of the present disclosure;

FIG. 5C is a cross-sectional view taken along line VC-VC of FIG. 5B;

FIG. 6 is a flowchart showing a flow of operations when the centrifugal separation system performs centrifugal separation in accordance with embodiments of the present disclosure;

FIG. 7A is a perspective view showing a first state of a clamp according to a second embodiment of the present disclosure;

FIG. 7B is a perspective view showing a second state of the clamp shown in FIG. 7A;

FIG. 8A is a front view showing another exemplary configuration of the clamp according to the second embodiment of the present disclosure;

FIG. 8B is a front view showing yet another exemplary configuration of the clamp according to the second embodiment of the present disclosure;

FIG. 9 is a perspective view showing a clamp according to a third embodiment of the present disclosure;

FIG. 10 is a perspective view showing a clamp according to a fourth embodiment of the present disclosure;

FIG. 11A is a perspective view showing a second state of a clamp according to a fifth embodiment of the present disclosure;

FIG. 11B is a cross-sectional view taken along line XIB-XIB of FIG. 11A;

FIG. 11C is a cross-sectional view taken along line XIC-XIC of FIG. 11A;

FIG. 12 is a perspective view showing a second state of a clamp according to a sixth embodiment of the present disclosure;

FIG. 13A is a front view showing a first state of a clamp according to a seventh embodiment of the present disclosure;

FIG. 13B is a front view showing a second state of the clamp according to the seventh embodiment of the present disclosure; and

FIG. 14 is a front view of a clamp according to an eighth embodiment of the present disclosure.

### DETAILED DESCRIPTION

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The ensuing description provides embodiments only, and is not intended to limit the scope, applicability, or configuration of the claims. Rather, the ensuing description will provide those skilled in the art with an enabling description for implementing the described embodiments. It being understood that various changes may be made in the function and arrangement of elements without departing from the spirit and scope of the appended claims.

Various aspects of the present disclosure will be described herein with reference to drawings that may be schematic illustrations of idealized configurations.

Embodiments of the present disclosure will be presented and described in detail below with reference to the accompanying drawings.

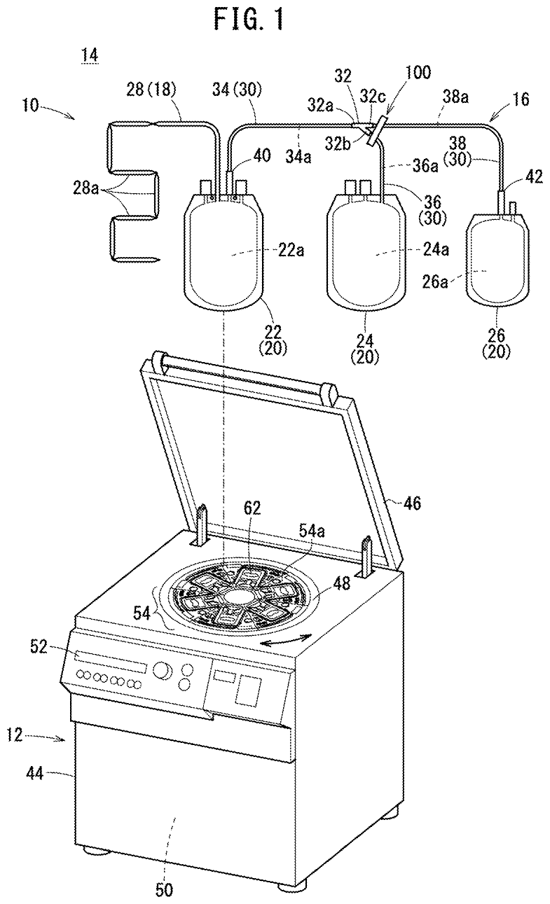

A blood bag system 10 according to a first embodiment of the present disclosure is set in a centrifugal separation and transferring device 12 as shown in FIG. 1, by a user such as a medical worker, technician, and/or the like. The centrifugal separation and transferring device 12 subjects the blood of the blood bag system 10 to centrifugal separation to thereby generate a plurality of types of blood components, and transfers and stores the generated blood components in appropriate bags 20. Hereinafter, a configuration including the blood bag system 10 and the centrifugal separation and transferring device 12 will be referred to as a centrifugal separation system 14.

The blood bag system 10 includes a non-illustrated preprocessing unit used for blood collection prior to centrifugal separation, a separation processing unit 16 that generates the blood components by way of centrifugal separation and separately stores the blood components, and a relay tube 18 connecting the preprocessing unit and the separation processing unit 16 together. Prior to centrifugal separation, in a state in which the relay tube 18 connected to the preprocessing unit is disconnected as shown in FIG. 1, the separation processing unit 16 is set in the centrifugal separation and transferring device 12.

The preprocessing unit of the blood bag system 10 collects whole blood from a blood donor, and further, removes blood components such as leukocytes from the whole blood. Therefore, the preprocessing unit includes a blood collection needle, a blood collection bag, an initial flow blood collection bag, and a filter (for example, a leukocyte removal filter) and the like, none of which are shown, and is constituted by connecting the respective members by a plurality of tubes 30. More specifically, the preprocessing unit stores, in the initial flow blood collection bag, initial blood from among the whole blood of the donor collected through the blood collection needle, and thereafter, stores the remaining blood in the blood collection bag. Furthermore, by the whole blood stored in the blood collection bag passing through the filter, the preprocessing unit removes the leukocytes in the filter. Then, after the leukocytes have been removed, the blood is transferred into the separation processing unit 16 that is connected to the filter via the relay tube 18.

The separation processing unit 16 of the blood bag system 10 includes a plurality of bags 20 (e.g., a blood bag 22, a PPP bag 24, and a chemical solution bag 26), and is constituted by connecting the respective bags 20 by the plurality of tubes 30.

The blood bag 22 is directly connected to the relay tube 18 that is connected with the preprocessing unit, in an initial state of the blood bag system 10 before use. The blood bag 22 is a first bag having a first storage space 22a for storing blood from which leukocytes have been removed by passing through the filter at the time of blood collection. The blood bag 22 is set in the centrifugal separation and transferring device 12, and is applied with a centrifugal force by operation of the centrifugal separation and transferring device 12. Consequently, the removed blood in the blood bag 22 is subjected to centrifugal separation into blood components such as platelet poor plasma (PPP), concentrated red blood cells (RBC), and the like having different specific gravities. In addition, after the centrifugal separation, by transferring the PPP under operation of the centrifugal separation and transferring device 12, the blood bag 22 stores only the remaining RBC.

The PPP bag 24 is a second bag (e.g., plasma bag) that stores, in a second storage space 24a, the PPP that is supplied thereto from the blood bag 22. On the other hand, the chemical solution bag 26 is a third bag that accommodates beforehand, in a third storage space 26a, a red blood cell preservative solution (hereinafter, referred to as an additive solution) such as a Mannitol-Adenine-Phosphate (MAP) solution, a Saline Adenine Glucose Mannitol (SAGM) solution, OPTISOL solution, and/or the like.

Further, prior to the separation processing unit 16 being set in the centrifugal separation and transferring device 12, the relay tube 18 is sealed at predetermined intervals to thereby form tube segments 28.

The plurality of tubes 30 of the separation processing unit 16 are branched in multiple directions via a branching connector 32 (Y-type connector: branching portion). In greater detail, the plurality of tubes 30 include a first tube 34 that connects the blood bag 22 and the branching connector 32, a second tube 36 that connects the PPP bag 24 and the branching connector 32, and a third tube 38 that connects the chemical solution bag 26 and the branching connector 32. The first tube 34 includes a first flow path 34a, the second tube 36 includes a second flow path 36a, and the third tube 38 includes a third flow path 38a.

Further, the branching connector 32 includes a first port 32a to which the first tube 34 is connected, a second port 32b to which the second tube 36 is connected, and a third port 32c to which the third tube 38 is connected, and the first to third ports 32a to 32c are integrally molded. The first to third ports 32a to 32c communicate with each other by a non-illustrated internal flow path, whereby the respective flow paths of the first to third tubes 34, 36, and 38 are placed in communication with each other.

At an end portion of the first tube 34 on the side of the blood bag 22, there is provided a sealing member 40 that is capable of being broken, and includes an internal flow path that opens accompanying breaking of the tube by the user. Similarly, a sealing member 42 that is capable of being broken is provided at an end portion of the third tube 38 on the side of the chemical solution bag 26. The sealing members 40 and 42 block the first flow path 34a and the third flow path 38a respectively until the breaking operation is performed, and serve to prevent the blood inside the blood bag 22 and the additive solution inside the chemical solution bag 26 from being transferred into the other bags 20.

Further, the blood bag system 10 is equipped with a single clamp 100 that is mounted over the second tube 36 and the third tube 38 at positions in the vicinity of the branching connector 32. The clamp 100 is configured to be capable of switching the second flow path 36a of the second tube 36 and the third flow path 38a of the third tube 38 from an open state to a blocked state. A description will be given in detail later concerning the specific configuration of the clamp 100.

On the other hand, the centrifugal separation and transferring device 12 in which the separation processing unit 16 of the blood bag system 10 is set is equipped with a box-shaped base body 44, a lid 46 that is capable of opening and closing an upper surface of the base body 44, and a centrifugal drum 48 provided on the base body 44. Further, a non-illustrated motor for causing the centrifugal drum 48 to rotate, a control unit 50 for controlling operations of the centrifugal separation and transferring device 12, and an operation display unit 52 in order for the user to confirm and carry out operations are provided on the base body 44 of the centrifugal separation and transferring device 12.

The centrifugal drum 48 includes a plurality of (six) unit setting areas 54 in which the separation processing unit 16 can be set. The height of one of the unit setting areas 54 is greater than a longitudinal length of the bags 20, and is set within a range of 60 degrees with respect to the center of rotation of the centrifugal drum 48. Stated another way, the centrifugal drum 48, which may be configured as an annular structural member, is constituted by arranging the six unit setting areas 54 alongside one another without gaps therebetween along a circumferential direction.

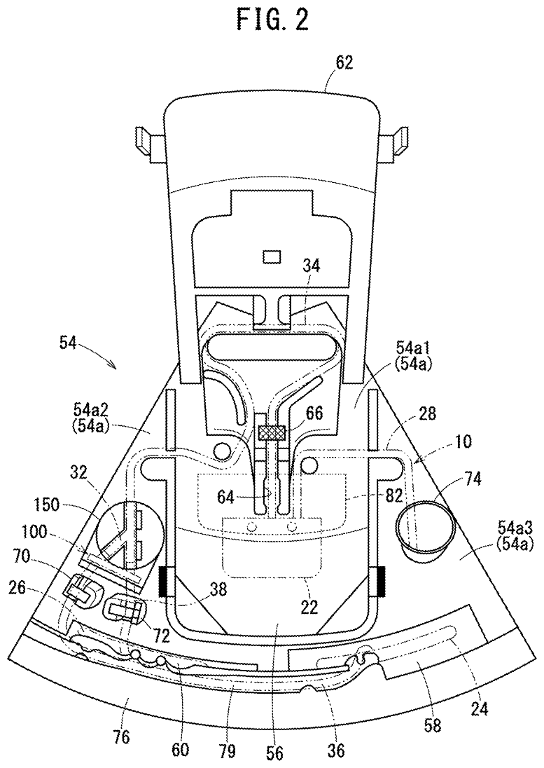

As shown in FIG. 2, the unit setting area 54 is equipped, at positions on an outer side in a radial direction of the centrifugal drum 48, with a blood bag pocket 56 in which the blood bag 22 is accommodated, a PPP bag pocket 58 in which the PPP bag 24 is accommodated, and a chemical solution bag pocket 60 in which the chemical solution bag 26 is accommodated.

The blood bag pocket 56 is provided in a central portion in the circumferential direction of the unit setting area 54, and has a volume that is greater than that of the PPP bag pocket 58 and the chemical solution bag pocket 60. The PPP bag pocket 58 and the chemical solution bag pocket 60 are disposed alongside one another in the circumferential direction, on a more outer side in the radial direction than the blood bag pocket 56.

Further, an upper surface 54a of the unit setting area 54 is configured such that the plurality of tubes 30 of the blood bag system 10 are arranged and retained thereon. On the upper surface 54a, the first tube 34 and one portion of the tube segments 28 are arranged in a central region 54a1 on a more inner side in the radial direction than the blood bag pocket 56. Further, in the central region 54a1, a lid 62 is provided that opens and closes an opening of the blood bag pocket 56. Furthermore, a portion of a breaking member 64 that serves to break the sealing member 40, and a sensor 66 that detects a state of the blood flowing through the first tube 34 are disposed at a position where the first tube 34 that is covered by the lid 62 is arranged.

In a left region 54a2 of the upper surface 54a, one portion of the first tube 34 after having passed through the central region 54a1, the branching connector 32, the second tube 36, and one portion of the third tube 38 are arranged. In the left region 54a2, a holder 150 for retaining the branching connector 32, a PPP clamp 70 for opening and closing the second flow path 36a of the second tube 36, and a chemical solution clamp 72 for opening and closing the third flow path 38a of the third tube 38 are provided.

In a right region 54a3 of the upper surface 54a, a segment pocket 74 is provided in which a plurality of hermetically sealed tubes 28a (see, e.g., FIG. 1) of the tube segments 28 are accommodated.

Furthermore, a tube retaining member 76 that projects upwardly from the upper surface 54a is provided on a more outer side in the radial direction than the PPP bag pocket 58 and the chemical solution bag pocket 60 in the unit setting area 54. The tube retaining member 76 includes a guide groove portion 79 for allowing the second tube 36 to be arranged therein.

Further, the unit setting area 54 is equipped with a slider 82 that presses on the blood bag 22 following completion of centrifugal separation, on an inner side in the radial direction of the blood bag pocket 56. The slider 82 advances and retracts along the radial direction of the centrifugal drum 48 under the control of the control unit 50 (see, e.g., FIG. 1).

In the above-described unit setting area 54, the user places, in the blood bag pocket 56, the blood bag 22 in which the removed blood is stored, places the empty PPP bag 24 in the PPP bag pocket 58, and places, in the chemical solution bag pocket 60, the chemical solution bag 26 in which the chemical solution is stored, respectively. In addition, in the unit setting area 54, the removed blood of the blood bag 22 is subjected to centrifugal separation by rotation of the centrifugal drum 48, and after centrifugal separation is completed, the slider 82 is advanced and presses on the blood bag 22. Consequently, the PPP, that is generated by centrifugal separation in the blood bag 22, is transferred into the PPP bag 24, whereas the RBC remains in the blood bag 22 after the PPP has been transferred. Following transfer of the PPP, the blood bag system 10 is taken out from the centrifugal separation and transferring device 12 by the user.

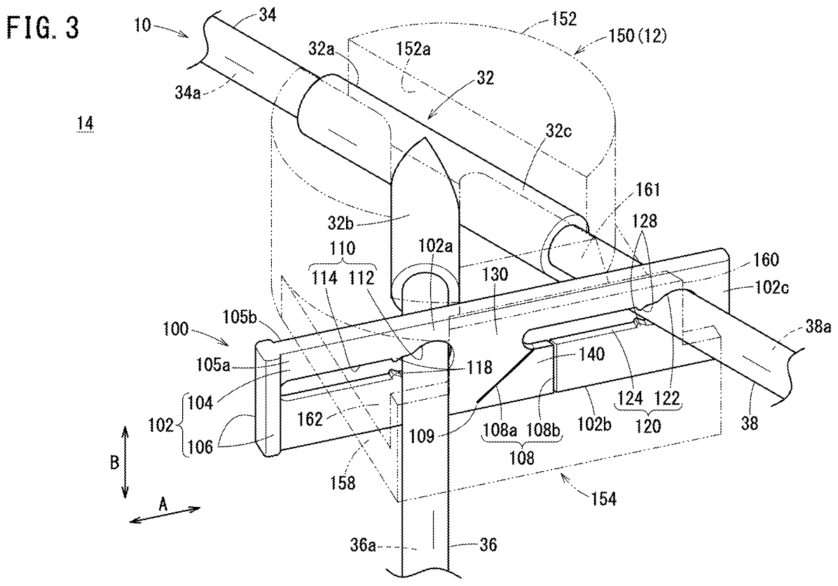

Next, a description is provided with reference to FIG. 3 concerning the clamp 100 of the blood bag system 10 according to the present embodiment.

As described above, the clamp 100 is mounted over the second tube 36 and the third tube 38 of the blood bag system 10, and is configured to enable the second flow path 36a and the third flow path 38a to be switched from an open state to a blocked state. Furthermore, the clamp 100 is configured in a manner so as to open the third flow path 38a of the third tube 38, that is in a blocked state, by being subjected to a deforming operation by the user after having been taken out from the centrifugal separation and transferring device 12.

More specifically, the clamp 100 comprises a plate-shaped main body 102 constituted by a resin material or a metal material. In addition, the clamp 100 includes a first insertion hole 110 that penetrates, or passes, through the main body 102 and in which the second tube 36 is arranged, and a second insertion hole 120 that penetrates, or passes, through the main body 102 and in which the third tube 38 is arranged.

The main body 102 is configured so as to possess a higher rigidity (a greater elastic modulus and hardness) than the tubes 30. The main body 102 includes a flat plate-shaped base 104 that exhibits a rectangular shape when viewed from the front, and flange portions 106 provided on one end side in a longitudinal direction (horizontal direction: the direction of the arrow A) of the base 104. The base 104 and the flange portions 106 are integrally molded from the same material.

When the blood bag system 10 is used, the base 104 constitutes a portion that is placed in direct contact with the second tube 36 and the third tube 38, and switches between opening and blocking of the second flow path 36a and the third flow path 38a. The first insertion hole 110 and the second insertion hole 120 penetrate, or pass, through a first surface 105a and a second surface 105b that make up the flat plate-shaped surface of the base 104.

The base 104 has an appropriate thickness in order to constitute an edge portion that is capable of being placed in surface contact with the outer circumferential surface of the second tube 36 and the outer circumferential surface of the third tube 38. The thickness of the base 104 is not particularly limited, but may be set, for example, on the order of 0.5 mm to 2 mm.

Apart from the one end in the longitudinal direction where the flange portions 106 are provided on the main body 102, the first surface 105a and the second surface 105b of the base 104 are continuous outer peripheral portions. Hereinafter, among the pair of long sides that extend along the longitudinal direction of the main body 102, the long side positioned on the upper side when set in the holder 150 is referred to as an upper side 102a, and the long side positioned on the lower side when set in the holder 150 is referred to as a lower side 102b. Further, on the base 104, a short side positioned at the other end in the longitudinal direction, that is an end portion on an opposite side to the flange portions 106, is referred to as a lateral side 102c.

The flange portions 106 are provided in a pair on both sides in the thickness direction of the base 104, project a short distance along the thickness direction, and extend in a lateral direction (vertical direction: the direction of the arrow B). The flange portions 106 make it easier for the operator to contact the clamp 100 with the fingers when pushing the clamp 100.

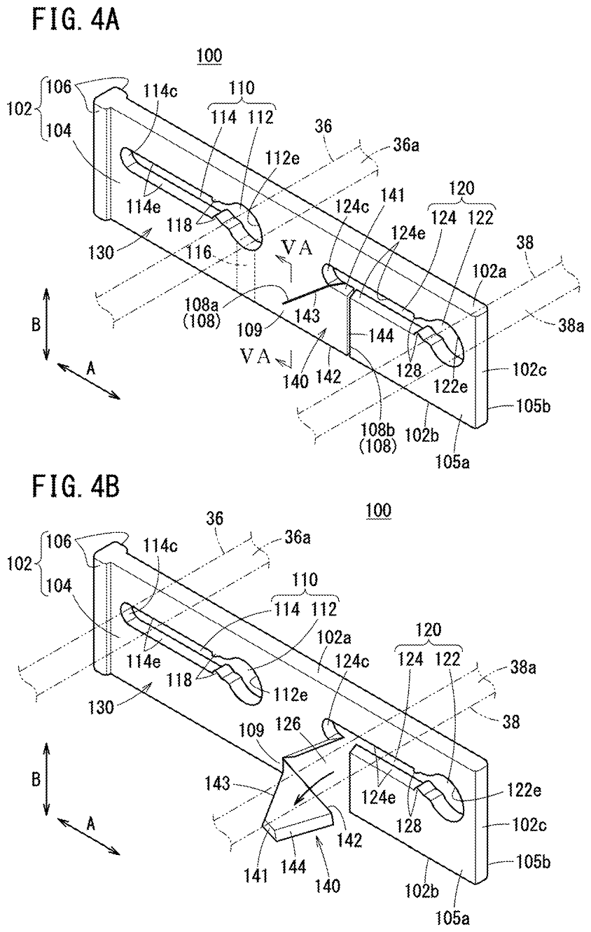

As shown in FIGS. 3 and 4A, the first insertion hole 110 and the second insertion hole 120 are formed by edge portions included on an inner side of the base 104. The first insertion hole 110 and the second insertion hole 120 are disposed side by side in the center in the lateral direction of the main body 102. The first insertion hole 110 and the second insertion hole 120 extend together along the longitudinal direction of the main body 102. More specifically, the first insertion hole 110 and the second insertion hole 120 are arranged in a row alongside one another, and extend in the same direction.

The first insertion hole 110 includes a first opening portion 112 that places the second flow path 36a of the second tube 36 in an open state, and a first blocking portion 114 that is connected with the first opening portion 112 and places the second flow path 36a of the second tube 36 in a blocked state. Moreover, the first insertion hole 110 may be equipped with a tube introducing portion 116 (refer to the dotted line shown in FIG. 4A) that is capable of introducing the second tube 36 from the exterior of the main body 102 into the first opening portion 112.

The first opening portion 112 is disposed in the vicinity of the center in the longitudinal direction of the main body 102. The first opening portion 112 is formed as a substantially perfect circular shaped space by a first arcuate edge portion 112e of the main body 102, and has a diameter greater than or equal to the outer diameter of the second tube 36. For example, the diameter of the first opening portion 112 is set on the order of 1.0 to 1.5 times the outer diameter of the second tube 36.

The first blocking portion 114 extends from the first opening portion 112 toward the flange portions 106 of the main body 102. The first blocking portion 114 is formed as a horizontal elongate space by a pair of first straight edge portions 114e extending in the longitudinal direction of the base 104, and with a width that is narrower than the outer diameter of the second tube 36. When the second tube 36 is moved into the first blocking portion 114, the pair of first straight edge portions 114e act to block the second flow path 36a of the second tube 36 by pressing inwardly on the outer circumferential surface of the second tube 36. In order for the second tube 36 to be easily inserted from the first opening portion 112 into the first blocking portion 114, the first insertion hole 110 may have a configuration in which the width between the pair of first straight edge portions 114e is narrowed in a tapered shape so that the edge portions come into closer proximity from the first opening portion 112 toward the inner back side of the first blocking portion 114.

One end side in the longitudinal direction of the first blocking portion 114 extends to the vicinity of the flange portions 106, and is closed by a semicircular shaped first blocking edge portion 114c. More specifically, in the first blocking portion 114, a portion thereof except for a portion connected with the first opening portion 112 is surrounded by the main body 102. Accordingly, the first insertion hole 110 is configured in a manner so that the second tube 36 is not detached from the first blocking portion 114 directly toward the exterior of the main body 102.

Further, the first blocking portion 114 is equipped with a pair of first protrusions 118 that project from the pair of first straight edge portions 114e in directions to approach toward each other. The pair of first protrusions 118 are disposed at positions in the vicinity of the first opening portion 112. The pair of first protrusions 118 are formed in a right triangular shape, and allow the second tube 36 to be moved from the first opening portion 112 into the first blocking portion 114, while preventing movement of the second tube 36 from the first blocking portion 114 into the first opening portion 112. Moreover, the first protrusion 118 may be provided on only one of the pair of first straight edge portions 114e. Moreover, the first protrusions 118 may be provided at a boundary between the first opening portion 112 and the first blocking portion 114.

On the other hand, the second insertion hole 120 includes a second opening portion 122 that places the third flow path 38a of the third tube 38 in an open state, and a second blocking portion 124 that is connected with the second opening portion 122 and places the third flow path 38a of the third tube 38 in a blocked state.

The second opening portion 122 is provided in the vicinity of the lateral side 102c of the main body 102. An interval, or distance, between a central part of the first opening portion 112 and a central part of the second opening portion 122 is set to a dimension that is one half the length in the longitudinal direction of the main body 102. The second opening portion 122 is formed as a substantially perfect circular shaped space by a second arcuate edge portion 122e of the main body 102, and has a diameter greater than or equal to the outer diameter of the third tube 38. For example, the diameter of the second opening portion 122 is set on the order of 1.0 to 1.5 times the outer diameter of the third tube 38.

The second blocking portion 124 extends from the second opening portion 122 toward a center in the longitudinal direction of the main body 102. The second blocking portion 124 is formed as a horizontal elongate space by a pair of second straight edge portions 124e extending in the longitudinal direction of the base 104 and with a width that is narrower than the outer diameter of the third tube 38, and a semicircular shaped second blocking edge portion 124c on one end side in the longitudinal direction. In the present embodiment, the width between the pair of second straight edge portions 124e is the same as the width between the pair of first straight edge portions 114e. When the third tube 38 is moved into the second blocking portion 124, the pair of second straight edge portions 124e act to block the third flow path 38a of the third tube 38 by pressing inwardly on the outer circumferential surface of the third tube 38. In order for the third tube 38 to be easily inserted from the second opening portion 122 into the second blocking portion 124, the second insertion hole 120 may have a configuration in which the width between the pair of second straight edge portions 124e is narrowed in a tapered shape so that the edge portions come into closer proximity from the second opening portion 122 toward the inner back side of the second blocking portion 124. Further, the second blocking portion 124, similar to the first blocking portion 114, is equipped with a pair of second protrusions 128 that project from the pair of second straight edge portions 124e in directions to approach toward each other. The pair of second protrusions 128 are provided at positions in the vicinity of the second opening portion 122, or at a boundary between the second opening portion 122 and the second blocking portion 124.

In addition, by changing the form thereof from the first state to the second state, the main body 102 opens the third flow path 38a of the third tube 38. The first state is a state in which the third tube 38, that has been moved from the second opening portion 122 into the second blocking portion 124, is restrained by the second blocking portion 124. The second state is a state in which the third tube 38 is capable of being moved from the second blocking portion 124 to a position that is a position other than the second opening portion 122 and in which the third flow path 38a of the third tube 38 is reopened.

In order to carry out deformation of the main body 102, the base 104 is configured to include a base portion 130 (e.g., a first site) that constitutes the majority of the base 104, and a piece portion 140 (e.g., a second site) that moves relatively with respect to the base portion 130. Further, the base 104 includes a pair of slits 108 (e.g., a first slit 108a and a second slit 108b) at a boundary portion between the base portion 130 and the piece portion 140, and includes a hinge portion 109 that connects the base portion 130 and the piece portion 140.

In the first state, the base portion 130 and the piece portion 140 are arranged flush with each other. Upon transitioning from the first state to the second state, as shown in FIG. 4B, the piece portion 140 is rotated about the hinge portion 109 relative to the base portion 130 along the thickness direction of the main body 102. At this time, the hinge portion 109 is either plastically deformed or elastically deformed. Consequently, in the second state, the base portion 130 and the piece portion 140 exhibit a non-plate-like shape (e.g., a non-planar form), and a communication passage 126, that is a space connected with the second blocking portion 124, is formed between the second blocking portion 124 and the lower side 102b.

As shown in FIGS. 3, 4A, and 4B, the base portion 130 substantially coincides with the rectangular shape of the base 104 when viewed from the front, and on the inner side thereof, the entirety of the first insertion hole 110, and a major portion except for one portion of the second insertion hole 120 are defined. The piece portion 140 defines at least a portion of the second insertion hole 120. The piece portion 140 according to the present embodiment is disposed in a substantially central region in the longitudinal direction of the base 104, and additionally, within a range from the center in the lateral direction to the lower side 102b, and is formed in a trapezoidal shape when viewed from the front.

More specifically, the piece portion 140 includes an upper bottom 141 that constitutes a part of the second straight edge portions 124e of the second blocking portion 124, and a lower bottom 142 that constitutes a part of the lower side 102b of the main body 102, and the lower bottom 142 is formed to be longer than the upper bottom 141. The length of the upper bottom 141 may be set to be roughly the same as the outer diameter of the third tube 38, for example.

Further, among a pair of legs (e.g., opposite sides) of the piece portion 140, a first leg 143 closer to the central side in the longitudinal direction of the main body 102 extends in an inclined manner from one end of the upper bottom 141 toward one end of the lower bottom 142, and the extended end thereof reaches the hinge portion 109. The first leg 143 forms the first slit 108a between the first leg 143 and an opposing side of the base portion 130 that is inclined in a similar manner. A second leg 144 of the piece portion 140 extends between the other end of the upper bottom 141 and the other end of the lower bottom 142 in the direction of the arrow B perpendicular to the direction in which the second blocking portion 124 extends. The second leg 144 forms the second slit 108b between the second leg 144 and an opposing side of the base portion 130 that extends similarly in the direction of the arrow B.

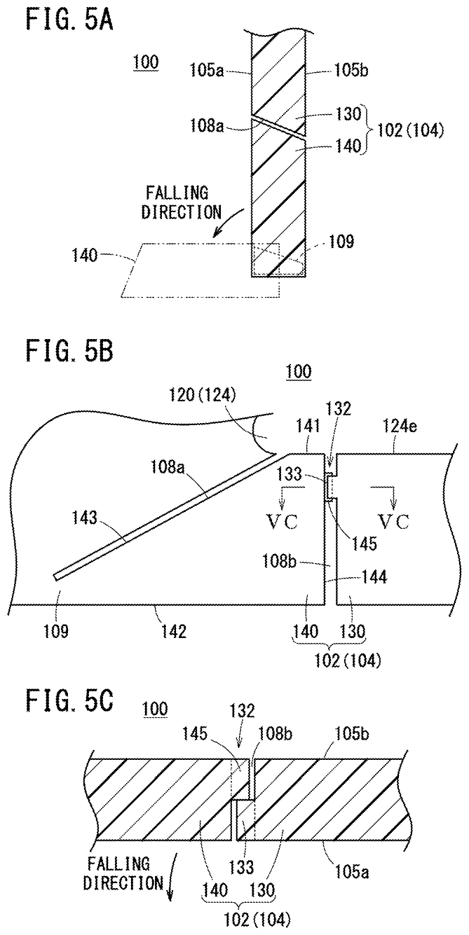

The first slit 108a is a first separation line penetrating, or passing, between the first surface 105a and the second surface 105b of the base 104 in the thickness direction. Further, as shown in FIG. 5A, an end portion of the first slit 108a on the side of the first surface 105a is formed to be high, whereas an end portion of the first slit 108a on the side of the second surface 105b is formed to be low, as viewed in cross-section along the thickness direction and the lateral direction of the main body 102. Stated otherwise, the cut of the first slit 108a in the thickness direction is inclined with respect to the planar direction of the main body 102 that is perpendicular to the thickness direction, in a manner so as to gradually decrease in height from the first surface 105a toward the second surface 105b. During the deformation, the first slit 108a, that is formed in this manner, induces the piece portion 140 to open toward the side of the first surface 105a, and conversely, restricts the piece portion 140 from opening toward the side of the second surface 105b.

Further, returning to FIG. 4A, the second slit 108b is a second separation line penetrating between the first surface 105a and the second surface 105b of the base 104 in the thickness direction. Unlike the first slit 108a, the second slit 108b extends parallel to the thickness direction of the main body 102.

Moreover, although the clamp 100 according to the present embodiment comprises the first slit 108a and the second slit 108b at the boundary between the base portion 130 and the piece portion 140, if the base portion 130 and the piece portion 140 are capable of being separated when the main body 102 is deformed, the base portion 130 and the piece portion 140 may be connected at the boundary. For example, the boundary between the base portion 130 and the piece portion 140 may be provided with a fragile, or frangible, portion (not shown) that is constituted by a wall portion that is thinner than the thickness of the base 104, perforations, an adhesive having a weak adhesive force, or the like, and that is broken upon being subjected to the deforming operation by the user. More specifically, the first and second separation lines are constituted by the slits 108, or alternatively, by fragile portions. In the first separation line and the second separation line, portions of the slits 108 in the direction of extension thereof may be connected with the fragile portions. The fragile portions may be used alternatively for both or either one of the first slit 108a and the second slit 108b.

Further, as shown in FIGS. 5B and 5C, the clamp 100 may be provided with a locking mechanism 132 in the second slit 108b, in order to prevent the piece portion 140 from unintentionally falling, or displacing, to the first surface 105a. For example, the locking mechanism 132 may have a configuration in which a base portion side hook 133 projecting from the base portion 130, and a piece portion side hook 145 projecting from the piece portion 140 are hooked onto each other. In this case, the base portion side hook 133 is formed over a range from the first surface 105a side of the base 104 to substantially the center in the thickness direction, and the piece portion side hook 145 is formed over a range from the second surface 105b side of the base 104 to substantially the center in the thickness direction. Consequently, in a falling, or displacement, direction in which the piece portion 140 falls, or displaces, toward the side of the first surface 105a, the piece portion side hook 145 and the base portion side hook 133 are hooked onto each other. The base portion side hook 133 and the piece portion side hook 145 are unlocked by an operating force from the user that causes the main body 102 to be deformed, thereby enabling the piece portion 140 to be moved.

Further, the hinge portion 109 that connects the base portion 130 and the piece portion 140 is positioned at a corner portion where the lower bottom 142 of the piece portion 140 and the first leg 143 are in proximity to each other. By being formed in a thin-walled fashion, the hinge portion 109 is configured to be capable of being plastically deformed or elastically deformed, in a manner so that the entirety of the hinge portion 109 is twisted. The hinge portion 109 is configured so as to be incapable of breaking, upon the piece portion 140 being rotated roughly 90 degrees. Alternatively, on the clamp 100, by the hinge portion 109 being broken due to deformation of the main body 102, the base portion 130 and the piece portion 140 may be separated mutually from each other.

As shown in FIG. 4B, in the second state in which the piece portion 140 has been deformed, the communication passage 126 is formed between the second blocking portion 124 and the lower side 102b. More specifically, when viewed from the front of the main body 102, the shape of the communication passage 126 coincides with the shape of the piece portion 140 including the slits 108. The communication passage 126 communicates with the second blocking portion 124, and makes it possible for the third tube 38, that is positioned in the second blocking portion 124, to move into the communication passage 126. In the third tube 38 that has been moved into the communication passage 126, the third flow path 38a is opened.

The communication passage 126 communicates with the exterior of the main body 102 from the lower side 102b of the main body 102. Therefore, the third tube 38 is capable of being detached to the exterior of the main body 102 through the communication passage 126. Even if the third tube 38 is detached, since the clamp 100 maintains the inserted state of the second tube 36 in the first insertion hole 110, the clamp 100 does not become disconnected from the blood bag system 10.

When the blood bag system 10 is set on the centrifugal separation and transferring device 12, the aforementioned clamp 100 is placed in the first state and is fixed to the holder 150 together with the branching connector 32. As shown in FIG. 3, the holder 150 includes a connector retaining member 152 in which the branching connector 32 is retained, and a clamp retaining member 154 in which the clamp 100 is retained in a slidable manner.

The connector retaining member 152 is formed in a cylindrical columnar shape, and includes a Y-shaped retaining groove 152a that is cut out to a predetermined depth from the upper end portion, and is configured to enable the branching connector 32 to be externally fitted therein. The clamp retaining member 154 includes a base plate 158 that extends from a lower part of the connector retaining member 152, a retaining plate 160 erected at a projecting end of the base plate 158, and an opposing wall 161 that faces toward the retaining plate 160. The clamp retaining member 154 supports the clamp 100 such that the clamp 100 can slide in a horizontal direction (in the direction of the arrow A), by way of an arrangement space 162 that is sandwiched between a pair of wall portions (e.g., the retaining plate 160 and the opposing wall 161).

When the above-described clamp 100 is manufactured, the main body 102, that is in the first state, is formed by injection molding or the like, separately from the bags 20 and the tubes 30. In addition, in a process of manufacturing the blood bag system 10, when the bags 20 and the tubes 30 are respectively connected, the second tube 36 and the third tube 38 are inserted through the main body 102, and thereafter, the second tube 36 and the third tube 38 are fixed to the branching connector 32. Consequently, the blood bag system 10 having the clamp 100 is formed.

The blood bag system 10 and the centrifugal separation system 14 according to the first embodiment are basically configured in the manner described above. Next, a description will be given below concerning operations thereof.

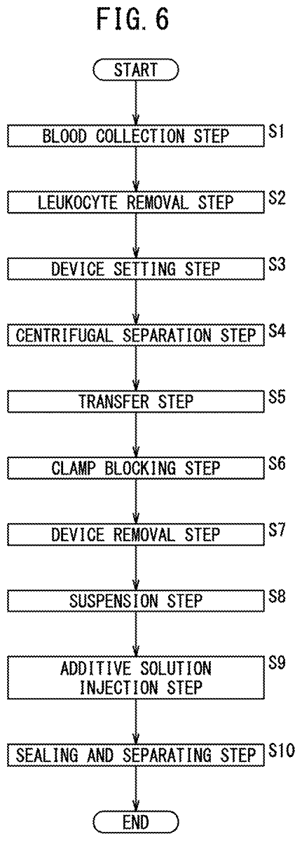

In the blood bag system 10, as shown in FIG. 6, a flow of operations, made up from a blood collection step (step S1), a leukocyte removal step (step S2), a device setting step (step S3), a centrifugal separation step (step S4), a transfer step (step S5), a clamp blocking step (step S6), a device removal step (step S7), a suspension step (step S8), an additive solution injection step (step S9), and a sealing and separating step (step S10), are executed sequentially by the user.

In the blood collection step, the user collects whole blood from a blood donor using a blood collection needle of a non-illustrated preprocessing unit, and stores the collected blood in a blood collection bag. In the leukocyte removal step, the user moves the whole blood from the blood collection bag into the blood bag 22, and at this time, by the whole blood passing through the filter, the removed blood from which leukocytes have been removed from the whole blood is stored in the blood bag 22.

In order for the removed blood to be subjected to centrifugal separation, in the device setting step, the user separates the separation processing unit 16 of the blood bag system 10 away from the preprocessing unit, and sets it in the centrifugal separation and transferring device 12. As shown in FIG. 2, the user accommodates the blood bag 22 in the blood bag pocket 56 of the unit setting area 54, and further, sets the first to third tubes 34, 36, and 38 along predetermined paths on the upper surface 54a of the unit setting area 54. The first tube 34 is arranged so as to proceed toward the left region 54a2, after being routed through the predetermined path in the central region 54a1. Further, the user passes the first to third tubes 34, 36, and 38 through the left region 54a2, and retains the branching connector 32 and the clamp 100 in the holder 150. The second tube 36 is arranged so as to extend through the left region 54a2 from the branching connector 32 to the outer side in the direction of centrifugation, and to pass through the PPP clamp 70. Furthermore, the second tube 36 is accommodated in the guide groove portion 79 of the tube retaining member 76 on an outer side in the radial direction of the chemical solution bag pocket 60. The third tube 38 also is arranged so as to extend through the left region 54a2 from the branching connector 32 to the outer side in the direction of centrifugation, and to pass through the chemical solution clamp 72. In addition, the user accommodates, in the PPP bag pocket 58, the PPP bag 24 that is connected to the second tube 36, and accommodates, in the chemical solution bag pocket 60, the chemical solution bag 26 that is connected to the third tube 38. Consequently, setting of the blood bag system 10 on the centrifugal separation and transferring device 12 is completed.

Further, at the time of setting, the clamp 100 is placed in the first state in which the base portion 130 and the piece portion 140 are arranged flush with each other. In a state in which the second tube 36 is arranged in the first opening portion 112, and the third tube 38 is arranged in the second opening portion 122, the user moves the clamp 100 to a position in the vicinity of the branching connector 32. In addition, as shown in FIG. 3, the user mounts the branching connector 32 in the connector retaining member 152 of the holder 150, while at the same time, inserts the clamp 100 into the arrangement space 162 between the retaining plate 160 and the opposing wall 161 of the holder 150. Accordingly, after the device setting step, the clamp 100 remains on standby on the unit setting area 54, in a state with the second flow path 36a of the second tube 36 and the third flow path 38a of the third tube 38 being open.

Thereafter, in the centrifugal separation step, the centrifugal separation and transferring device 12 causes the centrifugal drum 48 to rotate under the control of the control unit 50, thereby subjecting the removed blood from the blood bag 22 to centrifugal separation into blood components such as PPP, RBC, and the like having different specific gravities. During centrifugal separation, the second tube 36 and the third tube 38 are blocked by the PPP clamp 70 and the chemical solution clamp 72 (refer to FIG. 2), and flowing of the blood components therein is prevented.

Further, in the transfer step, the centrifugal separation and transferring device 12 opens only the PPP clamp 70 to press the blood bag 22 by the slider 82. Consequently, the PPP from the blood bag 22 flows sequentially through the first tube 34, the branching connector 32, and the second tube 36, and flows into the PPP bag 24.

After the transfer step, the user carries out the clamp blocking step to block the second tube 36 and the third tube 38 by the clamp 100 (see, e.g., FIG. 3). At this time, the user accesses the upper surface 54a of each of the unit setting areas 54, and causes the clamp 100 to slide with respect to the holder 150 toward the other end side in the longitudinal direction. During this sliding, by coming into contact with the retaining plate 160, the second tube 36 does not slide together with the clamp 100. Further, due to being connected to the second tube 36 via the branching connector 32, the third tube 38 is fixed on the retaining plate 160, and does not slide together with the clamp 100.

Thus, during this sliding, the first insertion hole 110 moves relatively with respect to the second tube 36, and the second tube 36 passes beyond the pair of first protrusions 118 and moves into the first blocking portion 114. Consequently, the first blocking portion 114 blocks the second flow path 36a of the second tube 36. Similarly, during the sliding, the second insertion hole 120 moves relatively with respect to the third tube 38, and the third tube 38 passes beyond the pair of second protrusions 128 and moves into the second blocking portion 124. Consequently, the second blocking portion 124 blocks the third flow path 38a of the third tube 38.

Next, in the device removal step, in the centrifugal separation and transferring device 12, by retracting the slider 82 and opening the chemical solution clamp 72 (see, e.g., FIG. 2), the blood bag system 10 can be detached from the centrifugal separation and transferring device 12, and the user takes out the blood bag system 10. Then, in the suspension step, the user suspends the chemical solution bag 26 on a non-illustrated stand, and furthermore, arranges the blood bag 22 on a lower side in the direction of gravity than the position of the chemical solution bag 26.

In the additive solution injection step, by opening the third tube 38 and breaking the sealing member 42, the user supplies the additive solution in the chemical solution bag 26 to the blood bag 22. At this time, due to deformation (e.g., transitioning from the first state to the second state) of the clamp 100, the user causes the third tube 38 to move from the second blocking portion 124. More specifically, as shown in FIGS. 4A and 4B, the user presses the third tube 38 against the upper bottom 141 of the piece portion 140. Consequently, the piece portion 140 is deformed so as to be expanded with respect to the base portion 130 in a direction perpendicular to the planar direction of the main body 102, thereby forming the communication passage 126.

At this time, due to the shape of the above-described first slit 108a (see, e.g., FIG. 5A), deformation of the piece portion 140 in a direction toward the first surface 105a is induced. Accompanying the above-described pressing and expanding operation (e.g., an operation of causing the third tube 38 to move in one direction) by the user, the third tube 38 is immediately moved into the communication passage 126. In particular, the piece portion 140 exists in a direction that differs from the extending direction in which the second blocking portion 124 extends. Therefore, the second tube 36 receives almost no influence of the force applied to the third tube 38 when the user detaches the third tube 38. As a result, the clamp 100 enables the third tube 38 to be moved, while suppressing movement of the second tube 36 from the first blocking portion 114 into the first opening portion 112. In addition, in the communication passage 126, the third flow path 38a of the third tube 38 is opened.

Further, the user causes the third tube 38 to be detached from the opening portion of the communication passage 126 on the lower side 102b. At this time, the hinge portion 109 of the clamp 100 continues to connect the base portion 130 and the piece portion 140, so that the piece portion 140 is not discarded or treated as refuse that is separated away from the base portion 130.

In the sealing and separating step after the additive solution injection step, the user seals and severs an appropriate position of the first tube 34. Consequently, the blood bag 22 is separated away from the blood bag system 10 in a state in which the RBC with the additive solution contained therein is stored. Similarly, on the second tube 36, the user seals and severs a portion thereof more on the side of the PPP bag 24 than the clamp 100. Consequently, the PPP bag 24 is separated away from the blood bag system 10 in a state with the PPP stored therein. The remaining parts of the blood bag system 10 including the clamp 100 are appropriately discarded.

The present disclosure is not limited to the above-described embodiment, and various modifications can be adopted in accordance with the essence and gist of the present disclosure. For example, the clamp 100 may have a configuration in which the tube introducing portion 116 is provided in the first insertion hole 110 and the piece portion 140 is elastically recoverable between the first state and the second state, whereby the clamp 100 may be later attached to the blood bag system 10 to which the bags 20 and the tubes 30 have initially been connected.

Further, for example, the clamp 100 may be equipped with a non-illustrated handle on the piece portion 140, in order to assist in deforming the piece portion 140 that is arranged flush along the planar direction of the base portion 130 in the first state. In this case, the handle preferably projects from the first surface 105a in the vicinity of the upper bottom 141.

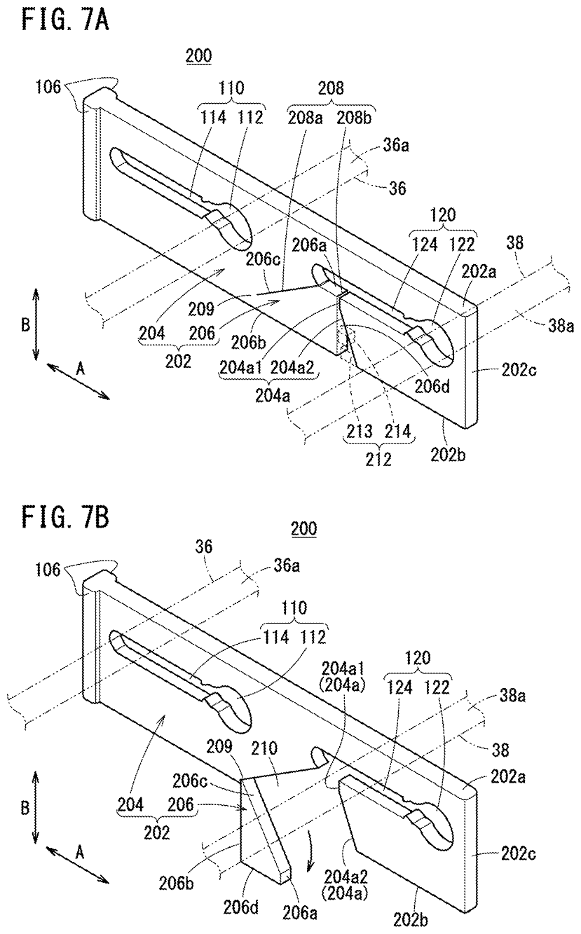

Next, with reference to FIGS. 7A and 7B, a description will be given concerning a clamp 200 according to a second embodiment of the present disclosure. A main body 202 of the clamp 200 differs from that of the aforementioned clamp 100 in that, during transitioning from the first state to the second state, a piece portion 206 (e.g., a second site) is deformed with respect to a base portion 204 (e.g., a first site) along a planar direction of the main body 202. In the following description, constituent elements having the same configuration or the same function as those of the above-described embodiment are designated by the same reference numerals, and detailed description of such features is omitted.

More specifically, the main body 202 is formed in a rectangular shape, and includes an upper side 202a and a lower side 202b on both sides thereof in the lateral direction, flange portions 106 on one end side in the longitudinal direction, and a lateral side 202c on another end side in the longitudinal direction. The piece portion 206 of the clamp 200 exhibits a trapezoidal shape in the same manner as the piece portion 140 of the clamp 100, and a pair of slits 208 (e.g., a first slit 208a and a second slit 208b) and a hinge portion 209 are provided at a boundary between the base portion 204 and the piece portion 206. However, the first slit 208a is not inclined with respect to the thickness direction, and further, the second slit 208b is formed in a triangular shape at a position lower than and separated slightly away from the second blocking portion 124. Moreover, even with such a clamp 200, a fragile portion may be applied to the first separation line (e.g., the first slit 208a) or the second separation line (e.g., the second slit 208b).

The piece portion 206 includes an upper bottom 206a, a lower bottom 206b, a first leg 206c, and a second leg 206d. The upper bottom 206a is formed to be shorter than the diameter of the third tube 38. Further, the first leg 206c is inclined with respect to the direction of the arrow B from an end portion of the upper bottom 206a to the hinge portion 209, whereas the second leg 206d extends along the direction of the arrow B perpendicular to the upper bottom 206a and the lower bottom 206b.

On the base portion 204, an opposing side 204a facing toward the second leg 206d of the piece portion 206 has a proximal side 204a1 that is close to the second blocking portion 124 (that is an upper part of the opposing side 204a) and is in close proximity to the piece portion 206. The proximal side 204a1 extends in a straight line shape along the direction of the arrow B. Further, the opposing side 204a on a lower side of the proximal side 204a1 is formed as an inclined side 204a2 that gradually separates away from the piece portion 206 toward the lower side 202b. More specifically, the second slit 208b includes an upper side portion where the proximal side 204a1 and the second leg 206d are in close proximity to each other, and a lower side portion where the inclined side 204a2 and the second leg 206d gradually separate away from each other toward the lower side 202b.

In the first state, the clamp 200 that is formed in this manner prevents the displacement of the piece portion 206 with respect to the base portion 204, by the second leg 206d of the piece portion 206 and the proximal side 204a1 of the base portion 204 being hooked onto each other. Further, since the length of the upper bottom 206a is shorter than the diameter of the third tube 38, the second blocking portion 124 can suitably squeeze the third tube 38 in the first state to block the third flow path 38a.

At the time of transitioning from the first state to the second state, by the user pressing the third tube 38 in a downward direction, hooking of the piece portion 206 with the proximal side 204a1 of the base portion 204 is released, and the piece portion 206 is rotated about the hinge portion 209. The piece portion 206 is deformed along a planar surface perpendicular to the thickness direction of the main body 202. Consequently, in the second state, a communication passage 210 is formed at the position where the piece portion 206 in the first state was located.

Moreover, apart from the shapes of the proximal side 204a1, the inclined side 204a2, and the second leg 206d, the clamp 200 may also be equipped with a locking mechanism 212, in order to prevent the piece portion 206 from being unintentionally rotated with respect to the base portion 204. For example, as shown by the dotted line in FIG. 7A, the locking mechanism 212 may be constituted by a base portion side hook 213 projecting from the inclined side 204a2, and a piece portion side hook 214 projecting from the second leg 206d on the upper side of the base portion side hook 213. Further, the clamp 200, similar to the clamp 100, may also have a configuration that is equipped with a handle in order to pull out the piece portion 206 in the first state.

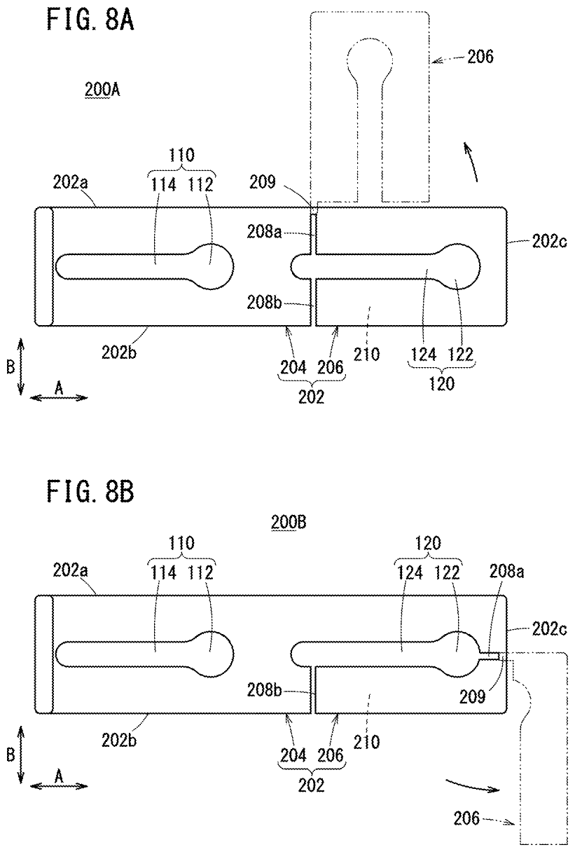

Furthermore, such a configuration in which the piece portion 206 is rotated along the planar surface of the main body 202 is not limited to providing the hinge portion 209 on the lower side 202b in the manner described above. As shown in FIG. 8A, a clamp 200A may be equipped with a first slit 208a and a second slit 208b extending from the second blocking portion 124 toward the upper side 202a and the lower side 202b, respectively, and may include a hinge portion 209 on the upper side 202a that is the extended end of the first slit 208a. In this case, the piece portion 206 is formed in a rectangular shape over substantially one half of the main body 202 including the second opening portion 122 and the second blocking portion 124.

In the first state, the lower side 202b is in close proximity to the base portion 204, whereby the clamp 200A makes the third tube 38 immovable from the second blocking portion 124. Upon transitioning from the first state to the second state, the piece portion 206 is rotated about the hinge portion 209, whereby the communication passage 210 (e.g., the outer side of the main body 202) that enables the third tube 38 to be moved from the second blocking portion 124, is formed at the location where the piece portion 206 in the first state was located.

Further, as shown in FIG. 8B, a clamp 200B may be configured to be equipped with a first slit 208a extending from the second opening portion 122 toward the lateral side 202c, and a second slit 208b extending from the second blocking portion 124 toward the lower side 202b, and to include a hinge portion 209 on the lateral side 202c that is the extended end of the first slit 208a. The piece portion 206 is formed in a rectangular shape over a range that extends from the center in the lateral direction to the lower side 202b, and extends from substantially the center in the longitudinal direction to the lateral side 202c.

In this case as well, in the first state, the lower side 202b is in close proximity to the base portion 204, whereby the clamp 200B makes the third tube 38 immovable from the second blocking portion 124. Upon transitioning from the first state to the second state, the piece portion 206 is rotated about the hinge portion 209, whereby the communication passage 210 (e.g., the outer side of the main body 202) that enables the third tube 38 to be moved from the second blocking portion 124, is formed at the location where the piece portion 206 in the first state was located.

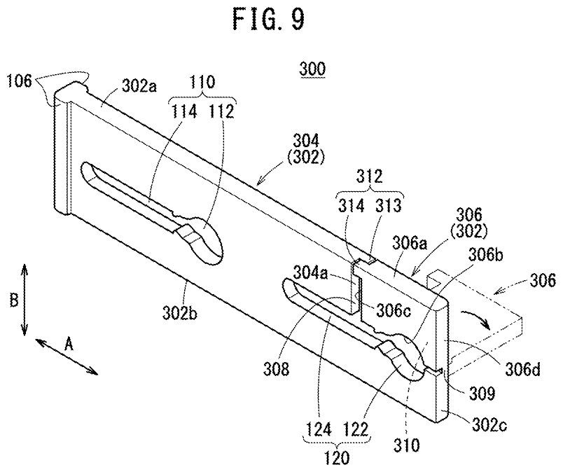

Next, with reference to FIG. 9, a description will be given concerning a clamp 300 according to a third embodiment of the present disclosure. A main body 302 of the clamp 300 differs from those of the aforementioned clamps 100 and 200 in that, during transitioning from the first state to the second state, a portion including the second opening portion 122 and the second blocking portion 124 is deformed in the thickness direction of the main body 302.

More specifically, the main body 302 is formed in a rectangular shape, and includes an upper side 302a and a lower side 302b on both sides thereof in the lateral direction, together with including the flange portions 106 on one end side in the longitudinal direction, and including a lateral side 302c on another end side in the longitudinal direction. In the main body 302, a piece portion 306 (e.g., a second site) is disposed on the other end side in the longitudinal direction and between the center in the lateral direction of the main body 302 and the upper side 302a, and a base portion 304 (e.g., a first site) constitutes the remaining portion other than the piece portion 306. A slit 308 and a hinge portion 309 are provided at a boundary between the base portion 304 and the piece portion 306.

The piece portion 306 includes an upper side area portion 306a constituting a part of the upper side 302a, and a lower side area portion 306b constituting a part of the second straight edge portion 124e of the second blocking portion 124 and the second arcuate edge portion 122e of the second opening portion 122. A first lateral side area portion 306c that extends along the direction of the arrow B is disposed between one end of the upper side area portion 306a and one end of the lower side area portion 306b in the longitudinal direction. A second lateral side area portion 306d, that constitutes a part of the lateral side 302c and extends along the direction of the arrow B, is disposed between another end of the upper side area portion 306a and another end of the lower side area portion 306b in the longitudinal direction.

The slit 308 is formed between the first lateral side area portion 306c of the piece portion 306, and an opposing side 304a of the base portion 304 facing toward the first lateral side area portion 306c. Moreover, a locking mechanism 312 may be provided in the slit 308 in order to prevent the piece portion 306 from unintentionally falling, or displacing, with respect to the base portion 304. For example, as shown in FIG. 9, the locking mechanism 312 may be constituted by a base portion side hook 313 and a piece portion side hook 314. The base portion side hook 313 projects from the opposing side 304a with a width that is one half of the thickness of the main body 302, and the piece portion side hook 314 projects from the first lateral side area portion 306c with a width that is one half of the thickness of the main body 302. The piece portion side hook 314 is positioned on an opposite side to the base portion side hook 313 in the thickness direction of the main body 302. A fragile portion may be applied to the separation line (e.g., the slit 308).

The hinge portion 309 extends in the direction of the arrow A between the second opening portion 122 and the lateral side 302c. The hinge portion 309 is constituted by making a portion of the main body 302 thin-walled, and connects the base portion 304 and the piece portion 306 on a side on which the piece portion 306 falls. The hinge portion 309 is configured to have a strength capable of maintaining the form of the first state in which the base portion 304 and the piece portion 306 are arranged flush with each other.

With the clamp 300 that is formed in the manner described above, the base portion 304 and the piece portion 306 are arranged flush with each other in the first state, whereby the third tube 38 is blocked while incapable of being detached from the second insertion hole 120. Accordingly, in the clamp 300, the third tube 38 is capable of being moved from the second opening portion 122 toward the second blocking portion 124.

When the main body 302 is deformed (e.g., transitions from the first state to the second state), the piece portion 306 is rotated about the hinge portion 309 with respect to the base portion 304. The piece portion 306 is deformed in the thickness direction of the main body 302. Consequently, in the second state, a communication passage 310 is formed at the position where the piece portion 306 in the first state was located.

The communication passage 310 is formed in the second blocking portion 124 in close proximity to the second opening portion 122. Therefore, the user is capable of recognizing the second insertion hole 120 without mistaking the respective shapes of the first insertion hole 110 and the second insertion hole 120. As a result, the user can easily move the third tube 38 from the second blocking portion 124 into the communication passage 310 and thereby open the third flow path 38a, and furthermore, is capable of allowing the third tube 38 to be detached from the main body 302.

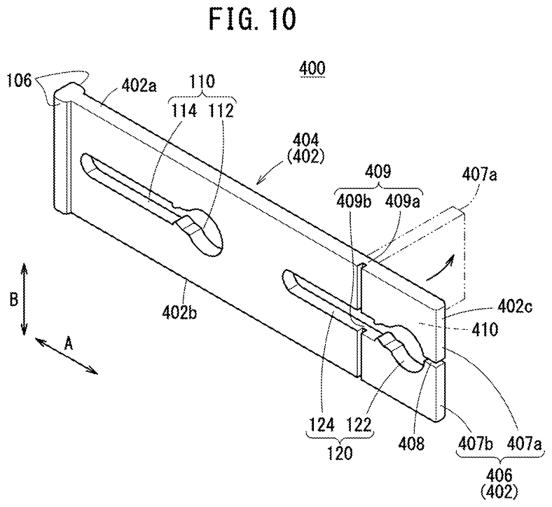

Next, with reference to FIG. 10, a description will be given concerning a clamp 400 according to a fourth embodiment of the present disclosure. A main body 402 of the clamp 400 differs from those of the aforementioned clamps 100, 200, and 300 in that, during transitioning from the first state to the second state, two portions in the vertical direction including the second opening portion 122 and the second blocking portion 124 are deformed in a direction perpendicular to the planar direction of the main body 402.

More specifically, the main body 402 is formed in a rectangular shape, and includes an upper side 402a and a lower side 402b on both sides thereof in the lateral direction, together with including the flange portions 106 on one end side in the longitudinal direction, and including a lateral side 402c on another end side in the longitudinal direction. In the main body 402, a base portion 404 is provided to span from the center in the longitudinal direction of the main body 402 to one end side in the longitudinal direction. Two piece portions 406 (e.g., second sites) are provided on the other end side in the longitudinal direction of the main body 402, and are constituted by a first divided piece 407a between the center in the lateral direction of the main body 402 and the upper side 402a, and a second divided piece 407b between the center in the lateral direction of the main body 402 and the lower side 402b.

A slit 408 is disposed at a boundary between the first divided piece 407a and the second divided piece 407b. A fragile portion may be applied to the boundary (e.g., the separation line) between the first divided piece 407a and the second divided piece 407b. Further, at a boundary between the base portion 404 (e.g., the first site) and the piece portions 406, hinge portions 409 (e.g., a first hinge portion 409a connecting the first divided piece 407a, and a second hinge portion 409b connecting the second divided piece 407b) are provided.

Also with the clamp 400 that is formed in the manner described above, the base portion 404 and the piece portions 406 are arranged flush with each other in the first state, whereby the third tube 38 of the second blocking portion 124 is restrained. When the main body 402 is deformed (e.g., transitions from the first state to the second state), for example, the first divided piece 407a is rotated about the first hinge portion 409a in the thickness direction of the main body 402. Consequently, in the second state, a communication passage 410 is formed at the position where the piece portion 406 in the first state was located.

The communication passage 410 is formed in the second blocking portion 124 in close proximity to the second opening portion 122, and communicates with the exterior of the main body 402 through the slit 408. In addition, since the second divided piece 407b is separated from the first divided piece 407a, the user can easily move the third tube 38 from the second blocking portion 124 into the communication passage 410 and thereby open the third flow path 38a, and furthermore, is capable of allowing the third tube 38 to be detached from the main body 402.

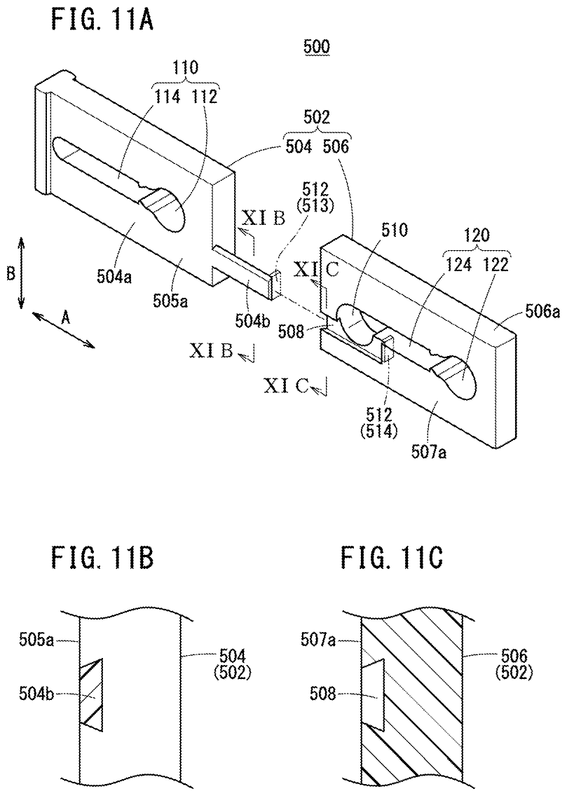

Next, with reference to FIGS. 11A and 11C, a description will be given concerning a clamp 500 according to a fifth embodiment of the present disclosure. A main body 502 of the clamp 500 differs from those of the aforementioned clamps 100, 200, 300, and 400, in that the main body 502 is configured to enable a first body 504 (e.g., a first site) having the first insertion hole 110, and a second body 506 (e.g., a second site) having the second insertion hole 120 to be separated away from each other. In this case, the main body 502 exhibits a form in which, in the first state, the first body 504 and the second body 506 are continuously integrated with each other, whereas in the second state, exhibits a form in which the first body 504 and the second body 506 are separated away from each other.

More specifically, the first body 504 makes up approximately one half of the main body 502 in proximity to one end in the longitudinal direction, and comprises a first base 504a having the first insertion hole 110 on an inner side thereof, and a projecting piece 504b that projects from the first base 504a. The second body 506 makes up approximately one half of the main body 502 in proximity to another end in the longitudinal direction, and comprises a second base 506a having the second insertion hole 120 on an inner side thereof. By the projecting piece 504b of the first body 504 being inserted in the second base 506a, the second base 506a retains the projecting piece 504b in a fitted manner.

The projecting piece 504b is disposed on the side of a first surface 505a of the first body 504, and is configured to have an appropriate thickness. As viewed in cross section along the thickness direction (see, e.g., FIG. 11B), the projecting piece 504b is formed in a trapezoidal shape expanding in the thickness direction from the first surface 505a. On the other hand, the second base 506a includes a fitting groove 508 on the side of a first surface 507a of the second body 506. As viewed in cross section along the thickness direction (see, e.g., FIG. 11C), the fitting groove 508 is formed in the same shape as the projecting piece 504b. Consequently, the projecting piece 504b and the second base 506a are fitted together with an appropriate frictional force, and further, coming out of the projecting piece 504b in the planar direction is prevented.

The second body 506 includes, as the second insertion hole 120, the second opening portion 122 and the second blocking portion 124, and furthermore, includes a reopening portion 510 that communicates with an end of the second blocking portion 124 on an opposite side to the second opening portion 122. The reopening portion 510 is formed as a substantially perfect circular shaped space (e.g., a space that is closed by the second body 506) having a diameter that enables the third flow path 38a of the third tube 38 to be opened. In addition, the reopening portion 510 is provided so as to overlap with the fitting groove 508 along the thickness direction of the second body 506. The reopening portion 510 may not be configured to overlap with the fitting groove 508 entirely, but may be configured such that a portion thereof does not overlap with the fitting groove 508. Consequently, even in the first state, the user is capable of easily recognizing the location (e.g., the second insertion hole 120) where the reopening portion 510 exists.

With the clamp 500 that is formed in the manner described above, in the first state, the projecting piece 504b of the first body 504 is inserted and fitted into the fitting groove 508 of the second body 506, whereby the first body 504 and the second body 506 form the plate-shaped main body 502 in which the first body 504 and the second body 506 are arranged flush with each other. In the first state, since the reopening portion 510 is closed by the projecting piece 504b, the third tube 38 is capable of being moved from the second opening portion 122 into the second blocking portion 124, and is restrained by the second blocking portion 124 and the third flow path 38a is blocked.

When the main body 502 is deformed (e.g., transitions from the first state to the second state), the user causes the first body 504 and the second body 506 to slide in directions to separate away from each other (in the direction of the arrow A), to thereby separate the first body 504 and the second body 506 away from each other. At this time, by the projecting piece 504b being pulled out from the fitting groove 508, the reopening portion 510 is exposed in the thickness direction of the second body 506. Consequently, the third tube 38 can be moved from the second blocking portion 124 into the reopening portion 510, and the third flow path 38a is opened in the reopening portion 510.

In addition, after having been moved into the reopening portion 510, the third tube 38 does not come out from the second body 506. More specifically, after the third flow path 38a of the third tube 38 has been opened, on the clamp 500, the first body 504 continues to be attached to the second tube 36, and the second body 506 continues to be attached to the third tube 38. Consequently, the clamp 500 is discarded integrally together with the blood bag system 10.

The configuration of the clamp 500 is not limited to the above-described configuration, and various shapes can be adopted therefor. For example, the projecting piece 504b and the fitting groove 508 may be provided as a pair (in plurality) on both surfaces of the first body 504 and the second body 506. Further, the clamp 500 may include a locking mechanism 512 for locking the first body 504 and the second body 506. For example, as shown by the two dot dashed line in FIG. 11A, the locking mechanism 512 may be constituted by a hook 513 that projects from a projecting end of the projecting piece 504b, and a recess 514 that is inwardly recessed from the fitting groove 508 of the second body 506.

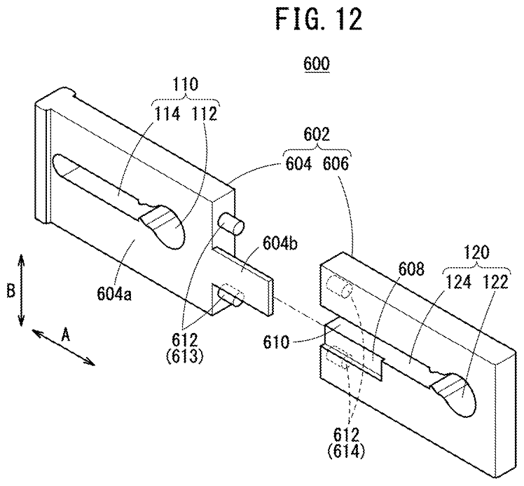

Next, with reference to FIG. 12, a description will be given concerning a clamp 600 according to a sixth embodiment of the present disclosure. A main body 602 of the clamp 600 differs from those of the aforementioned clamps 100, 200, 300, 400, and 500, in that the main body 602 is configured to enable a first body 604 (a first site) having the first insertion hole 110, and a second body 606 (a second site) having the second insertion hole 120 to be separated away from each other, and further, the third tube 38 is capable of being detached therefrom. More specifically, the main body 602 also exhibits a form in which the first body 604 and the second body 606 are continuously integrated with each other in the first state. In addition, in the second state, in a form in which the first body 604 and the second body 606 are separated away from each other, a communication passage 610 is formed that allows the third tube 38 to be detached from the second blocking portion 124.

The communication passage 610 is formed as a straight line shaped passage communicating with an end of the second blocking portion 124 on an opposing side to the second opening portion 122. The width of the communication passage 610 corresponds to the width of the second blocking portion 124. In the first state, the communication passage 610 is covered by a projecting piece 604b that projects from a base portion 604a of the first body 604. Further, a fitting groove 608 into which the projecting piece 604b is inserted and fitted is formed in the second body 606.

Furthermore, the clamp 600 includes a locking mechanism 612 that locks the first body 604 and the second body 606 in the first state. The locking mechanism 612 is constituted by a pair of fitting projections 613 provided on one of the first body 604 and the second body 606, and a pair of fitting recesses 614 provided on the other of the first body 604 and the second body 606.

With the clamp 600 that is formed in the manner described above, since the communication passage 610 is closed by the projecting piece 604b in the first state, the third tube 38 is moved from the second opening portion 122 into the second blocking portion 124, and the third flow path 38a is blocked. In addition, when the main body 602 is deformed (e.g., transitions from the first state to the second state), by the user separating the first body 604 and the second body 606, the projecting piece 604b is pulled out from the fitting groove 608, and the communication passage 610 is exposed. Consequently, the third tube 38 passes through the communication passage 610 from the second blocking portion 124, and can be detached from the second body 606, and due to such detachment, the third flow path 38a is opened. The second body 606 that has been detached from the third tube 38 is appropriately discarded by the user.

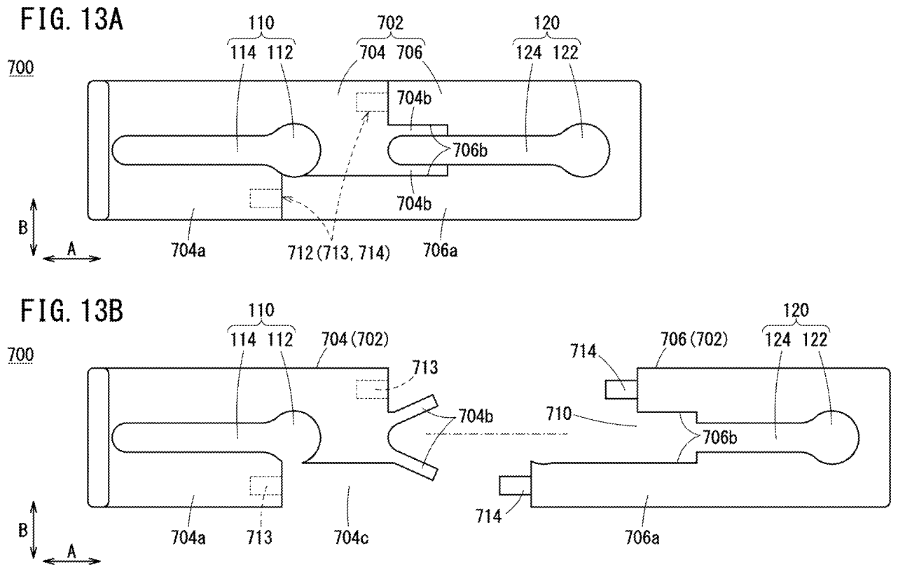

Next, with reference to FIGS. 13A and 13B, a description will be given concerning a clamp 700 according to a seventh embodiment of the present disclosure. Similar to the clamp 600, a main body 702 of the clamp 700 is configured to enable a first body 704 (e.g., a first site) having the first insertion hole 110, and a second body 706 (e.g., a second site) having the second insertion hole 120 to be separated away from each other, and further, the third tube 38 is capable of being detached therefrom. However, the clamp 700 is configured to enable the third tube 38 to be pulled out from the first body 704, at a time when the first body 704 and the second body 706 are separated away from each other.

More specifically, the first body 704 includes a first base 704a having the first insertion hole 110, and a pair of gripping pieces 704b, or arms, that project from the first base 704a. The pair of gripping pieces 704b include therebetween the second blocking edge portion 124c constituting part of the second blocking portion 124, and further, root portions thereof are swingably or hingedly connected to the first base 704a. The pair of gripping pieces 704b may be shaped so that the protruding ends thereof are greatly separated away from each other in the second state. Stated another way, the pair of gripping pieces 704b may be formed in the shape shown in FIG. 13B, and may deform into the shape shown in FIG. 13A when the first body 704 is connected, or locked, to the second body 706. Furthermore, the first body 704 includes a cutout 704c that communicates with the first opening portion 112. The cutout 704c constitutes the tube introducing portion 116 (see, e.g., FIG. 4A) capable of introducing the second tube 36 into the first insertion hole 110.

On the other hand, the second body 706 includes, on an inner side thereof, a part of the second blocking portion 124 and the second opening portion 122, and further, includes a projecting portion 706a at a location corresponding to the cutout 704c of the first body 704. In the second body 706, on one end side in the longitudinal direction of the second blocking portion 124, a pair of steps 706b, in which the pair of gripping pieces 704b are inserted and that make up the second blocking portion 124 together with the pair of gripping pieces 704b, are provided.

Further, the clamp 700 is equipped with a pair of fitting recesses 713 in the first body 704, while on the other hand, is equipped with a pair of fitting projections 714 in the second body 706. The pair of fitting recesses 713 and the pair of fitting protrusions 714 constitute a locking mechanism 712 that locks the first body 704 and the second body 706 in the first state.

In the main body 702 of the clamp 700 that is formed in the manner described above, the first body 704 and the second body 706 are connected and are continuous so as to be flush in the first state. In this case, the pair of gripping pieces 704b of the first body 704 are arranged on the steps 706b of the second body 706, and thereby form the second blocking portion 124 that is made up from a straight line shaped or a tapered space. Accordingly, the third tube 38 that is positioned in the second opening portion 122 can be smoothly moved toward the second blocking portion 124. In addition, when the clamp 700 is subjected to sliding (e.g., in the clamp blocking step), the third tube 38 is moved to a position where the third tube 38 is sandwiched between the pair of gripping pieces 704b.

When the main body 702 is deformed (e.g., transitions from the first state to the second state), the user causes the first body 704 and the second body 706 to slide in directions to separate away from each other (in the direction of the arrow A), to thereby separate the first body 704 and the second body 706 away from each other. At this time, the operation of pulling out the pair of gripping pieces 704b from the pair of steps 706b is performed, and the third tube 38 that is sandwiched between the pair of gripping pieces 704b is pulled out from the second body 706. More specifically, a communication passage 710 is formed between the first body 704 and the second body 706. By the third tube 38 being moved into the communication passage 710 (e.g., to the outer side of the main body 702), the third flow path 38a is opened.

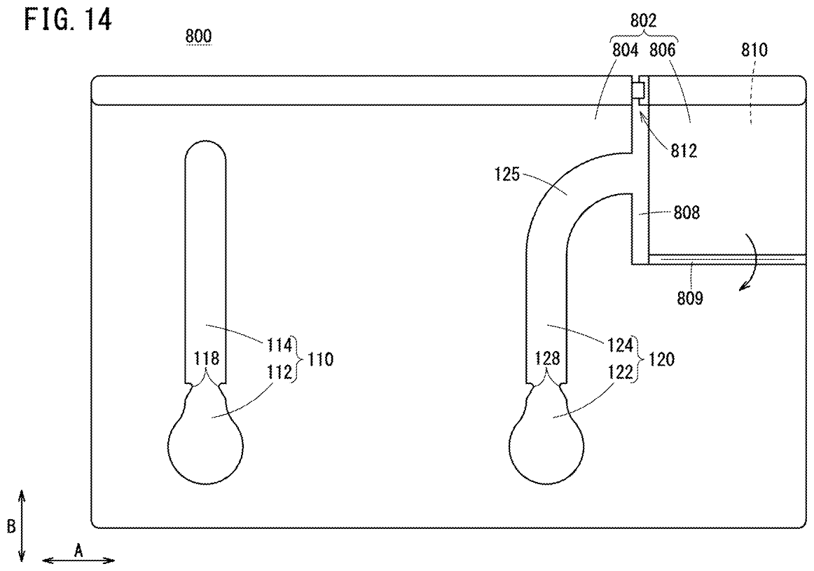

Next, with reference to FIG. 14, a description will be given concerning a clamp 800 according to an eighth embodiment of the present disclosure. The clamp 800 differs from those of the aforementioned clamps 100, 200, 300, 400, 500, 600, and 700, in that the first insertion hole 110 and the second insertion hole 120 extend in the lateral direction of a main body 802 (the direction of the arrow B). Moreover, the second insertion hole 120 includes a curved passage 125 in the second blocking portion 124, in order to distinguish it from the first insertion hole 110. The curved passage 125 is curved at 90 degrees with respect to a direction of extension of a lower side (e.g., the side of the second opening portion 122) of the second blocking portion 124 (the direction of the arrow A), and reaches the end portion.

Further, the main body 802 is constituted by a base portion 804 (e.g., a first site) constituting the first insertion hole 110 and the second insertion hole 120, and a piece portion 806 (e.g., a second site) disposed at an end portion of the second blocking portion 124. A slit 808 and a hinge portion 809 are provided at a boundary between the base portion 804 and the piece portion 806. The slit 808 communicates with the curved passage 125, and extends along the direction of the arrow B perpendicular to a tangential direction of an end portion of the curved passage 125 (the direction of the arrow A). Moreover, a fragile portion may be applied to a boundary between the base portion 804 and the piece portion 806. Further, the hinge portion 809 extends in the direction of the arrow A from the extended end portion of the slit 808.

In the first state, the clamp 800 that is formed in the manner described above exhibits a flat plate-like state in which the base portion 804 and the piece portion 806 are arranged flush with each other, and an end portion of the second blocking portion 124 is blocked by the piece portion 806. The clamp 800 is set in the holder 150 of the centrifugal separation and transferring device 12, and in the clamp blocking step (refer to FIG. 6), the clamp 800 is subjected to a sliding operation by the user in a downward direction (in the direction of the arrow B). Consequently, the second tube 36 is moved from the first opening portion 112 into the first blocking portion 114, and the third tube 38 is moved from the second opening portion 122 into the second blocking portion 124.

Further, in the additive solution injection step, the main body 802 of the clamp 800 is deformed by the user. Specifically, upon transitioning from the first state to the second state, the piece portion 806 is rotated about the hinge portion 809 in the thickness direction of the main body 802, whereby a communication passage 810 that communicates with the second blocking portion 124 and to which the third tube 38 can be moved is formed. The third tube 38 is detached from the main body 802 by moving into the communication passage 810, and the third flow path 38a is opened.

Moreover, as shown in FIG. 14, the clamp 800 is also preferably configured to include a locking mechanism 812 that locks the base portion 804 and the piece portion 806 in the first state.

Technical concepts, advantages, benefits, and effects provided by the above-described embodiments will be described below.

The first aspect of the present disclosure is characterized by the blood bag system 10, comprising the first bag (e.g., the blood bag 22) in which blood is accommodated, the second bag (e.g., the PPP bag 24) in which a blood component obtained by subjecting the blood inside the first bag to centrifugal separation is accommodated, the third bag (e.g., the chemical solution bag 26) in which the additive solution is accommodated, the first tube 34 connected to the first bag, the second tube 36 connected to the second bag, and the third tube 38 connected to the third bag, the first tube, the second tube, and the third tube being connected via the branching portion (e.g., the branching connector 32), the blood bag system further comprising the single clamp 100, 200, 300, 400, 500, 600, 700, or 800 that is mounted over the second tube and the third tube, wherein the clamp includes the main body 102, 202, 302, 402, 502, 602, 702, or 802, the first insertion hole 110 that penetrates, or passes, through the main body and in which the second tube is arranged, and the second insertion hole 120 that penetrates, or passes, through the main body and in which the third tube is arranged, the first insertion hole includes the first opening portion 112 that places the flow path (e.g., the second flow path 36a) of the second tube in an open state, and the first blocking portion 114 that is connected with the first opening portion and places the flow path of the second tube in a blocked state, and in the first blocking portion, a portion thereof except for a portion connected with the first opening portion is closed by the main body, and the second insertion hole includes the second opening portion 122 that places the flow path (e.g., the third flow path 38a) of the third tube in an open state, and the second blocking portion 124 that is connected with the second opening portion and places the flow path of the third tube in a blocked state, the main body is capable of being changed from the first state to the second state, in the first state, the third tube, that has been moved into the second blocking portion from the second opening portion, is restrained by the second blocking portion, and in the second state, the third tube is allowed to be moved from the second blocking portion to a position that is a position other than the second opening portion and in which the flow path of the third tube is reopened.

In accordance with the above-descried configuration, the blood bag system 10 is capable of efficiently blocking the flow paths of the two tubes 30 by a single clamp. Moreover, in the first insertion hole, the second tube is not detached from the first blocking portion. In contrast thereto, the second insertion hole, based on the deformation of the main body, allows the third tube to be moved to a position that is a position other than the second opening portion and in which the flow path of the third tube is reopened. Accordingly, in the blood bag system 10, a situation is avoided in which the two tubes are misidentified by the user, and opening of the flow path of the tube by mistake can be prevented. As a result, the blood bag system 10 can reduce mishandling of the system after centrifugal separation has been completed, and enables blood products to be accurately obtained.

Further, the main body includes the first site (e.g., the base portion 130, 204, 304, 404, or 804, the first body 504, 604, or 704) in which the first insertion hole is defined, and the second site (e.g., the piece portion 140, 206, 306, 406, or 806, the second body 506, 606, or 706) that is capable of being relatively displaced with respect to the first site and in which at least a portion of the second insertion hole is defined, and by the second site being relatively displaced with respect to the first site, the main body is changed from the first state to the second state. In accordance with these features, the blood bag system 10 is capable of more easily opening the flow path of the third tube, by transitioning from the first state to the second state due to the relative displacement of the second site with respect to the first site.

Further, the main body includes the hinge portion 109, 209, 309, 409, or 809 that connects the first site and the second site, by the second site being rotated about the hinge portion with respect to the first site, the main body is changed from the first state to the second state, and the main body, when changing from the first state to the second state, forms the communication passage 126, 210, 310, 410, or 810 that allows the third tube to be detached from the second blocking portion and moved to the exterior of the main body. In accordance with these features, by the second site being rotated about the hinge portion, the blood bag system 10 is capable of maintaining a satisfactory connection between the first site and the second site, and it is possible to prevent refuse, or discarded material, from being generated when such an operation is performed.

The main body is formed in a plate-like shape, and the second site can be rotated about the hinge portion with respect to the first site in a thickness direction of the main body. In accordance with these features, if the second site is configured to be rotated in the thickness direction of the main body, a large working space is not required. In addition, it is possible for the user to smoothly deform the second site.

The main body is formed in a plate-like shape, and the second site can be rotated about the hinge portion with respect to the first site along a planar surface perpendicular to a thickness direction of the main body. In accordance with this feature, even if the second site is rotated along the planar surface of the main body, it is possible for the user to smoothly deform the second site.

Further, the second site includes the inner end (e.g., the upper bottom 141 or 206a) forming a part of the edge portion (e.g., the second straight edge portions 124e) that forms the second blocking portion, and the main body includes the first separation line that is made up from the slit (e.g., the first slit 108a or 208a) or a fragile portion capable of being broken, and that lies adjacent to the inner end of the second site at one end thereof, and adjacent to the hinge portion at another end thereof, and the second separation line that is made up from the slit (e.g., the second slit 108b or 208b) or a fragile portion capable of being broken, and that lies adjacent to the inner end of the second site at one end thereof. In accordance with these features, the user is capable of easily displacing the second site along the first and second separation lines.

Further, the first separation line and the second separation line are both made up from the slits, the one end of the first separation line is connected with the edge portion, the one end of the second separation line is connected with the edge portion, and the other end of the second separation line is connected with an outer edge of the main body. In accordance with these features, the second portion becomes separated away from the first portion with the exception of the hinge portion, and it is possible for the user to easily perform an operation of displacing the second portion.

Further, the first blocking portion and the second blocking portion respectively extend from the first opening portion and the second opening portion mutually in the same direction, and the communication passage is formed in a direction that differs from the direction in which the second blocking portion extends. In accordance with these features, in an operation in which the user displaces the second site and moves the third tube, it is possible to prevent the second tube from being moved into the first opening portion.

Further, the second site is configured to be capable of separating away from the first site, and by the second site being separated away from the first site, the main body is changed from the first state to the second state. In accordance with these features, the user can more easily perform the operation of transitioning from the first state to the second state.

Further, the second insertion hole includes the reopening portion 510 that is connected with the second blocking portion at a position different from a position where the second blocking portion is connected with the second opening portion, and that allows the flow path of the third tube to be reopened, the first site includes the projecting piece 504b that restricts movement of the third tube from the second blocking portion into the reopening portion in the first state of the main body, and by the restriction of the third tube by the projecting piece being released when the second site is separated away from the first site, movement of the third tube into the reopening portion is permitted. In accordance with these features, after the first site and the second site have been separated, the user is capable easily moving the third tube into the reopening portion.

Further, when the second site is separated away from the first site, the second site forms the communication passage 610 or 710 that allows the third tube to be detached from the second blocking portion and moved to the exterior of the main body. In accordance with this feature, after the first site and the second site have been separated, the user is capable of easily detaching the third tube from the main body via the communication passage.

Further, the main body includes, at a boundary between the first site and the second site, the locking mechanism 132, 212, 312, 512, 612, 712, or 812 that locks the first portion and the second portion in the first state. In accordance with this feature, the clamp is capable of maintaining the first state satisfactorily until the user performs the operation of deforming the main body.

Further, the second aspect of the present disclosure is characterized by the clamp 100, 200, 300, 400, 500, 600, 700, or 800 that is mounted over the two tubes 30, the clamp comprising the main body 102, 202, 302, 402, 502, 602, 702, or 802, the first insertion hole 110 that penetrates, or passes, through the main body and in which one tube (e.g., the second tube 36) of the two tubes is arranged, and the second insertion hole 120 that penetrates, or passes, through the main body and in which the other tube (e.g., the third tube 38) of the two tubes is arranged, wherein the first insertion hole has the first opening portion 112 that places the flow path of the one tube in an open state, and the first blocking portion 114 that is connected with the first opening portion and places the flow path of the one tube in a blocked state, and in the first blocking portion, a portion thereof except for a portion connected with the first opening portion is closed by the main body, the second insertion hole has the second opening portion 122 that places the flow path of the other tube in an open state, and the second blocking portion 124 that is connected with the second opening portion and places the flow path of the other tube in a blocked state, the main body is capable of being changed from the first state to the second state, in the first state, the other tube, that has been moved into the second blocking portion from the second opening portion, is restrained by the second blocking portion, and in the second state, the other tube is allowed to be moved from the second blocking portion to a position that is a position other than the second opening portion and in which the flow path of the other tube is reopened. In accordance with these features, the clamp 100, 200, 300, 400, 500, 600, 700, or 800 is capable of efficiently blocking the flow paths of the two tubes 30, and preventing the flow path of the tube 30 from being opened by mistake.

References in the specification to “one embodiment,” “an embodiment,” “an example embodiment,” “some embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in conjunction with one embodiment, it is submitted that the description of such feature, structure, or characteristic may apply to any other embodiment unless so stated and/or except as will be readily apparent to one skilled in the art from the description. The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

It is to be appreciated that any feature described herein can be claimed in combination with any other feature(s) as described herein, regardless of whether the features come from the same described embodiment.

As used herein, the singular forms “a,” “an,” and “the” are intended to include the plural forms as well, unless the context clearly indicates otherwise. It will be further understood that the terms “include,” “including,” “includes,” “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. The term “and/or” includes any and all combinations of one or more of the associated listed items.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B, and C together. When each one of A, B, and C in the above expressions refers to an element, such as X, Y, and Z, or a class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

The terms “determine,” “calculate,” “compute,” and variations thereof, as used herein, are used interchangeably and include any type of methodology, process, mathematical operation, or technique.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and this disclosure.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

What is claimed is:

1. A blood bag system, comprising:
a first bag configured to accommodate blood;
a second bag configured to accommodate a blood component obtained by subjecting the blood inside the first bag to centrifugal separation;
a third bag configured to accommodate an additive solution;
a first tube connected to the first bag;
a second tube connected to the second bag; and
a third tube connected to the third bag,
the first tube, the second tube, and the third tube being connected via a branching portion,
the blood bag system further comprising a single clamp mounted over the second tube and the third tube, wherein the clamp includes:
a main body;
a first insertion hole that penetrates through the main body and in which the second tube is arranged; and
a second insertion hole that penetrates through the main body and in which the third tube is arranged,
the first insertion hole includes a first opening portion configured to place a flow path of the second tube in an open state, and a first blocking portion connected with the first opening portion and configured to place the flow path of the second tube in a blocked state, and in the first blocking portion, a portion thereof except for a portion connected with the first opening portion is closed by the main body,
the second insertion hole includes a second opening portion configured to place a flow path of the third tube in an open state, and a second blocking portion connected with the second opening portion and configured to place the flow path of the third tube in a blocked state,
the main body is configured to be changed from a first state to a second state,
in the first state, the third tube, that has been moved into the second blocking portion from the second opening portion, is restrained by the second blocking portion, and
in the second state, the third tube is allowed to be moved from the second blocking portion to a position that is a position other than the second opening portion and in which the flow path of the third tube is reopened.

2. The blood bag system according to claim 1, wherein:
the main body includes a first site in which the first insertion hole is defined, and a second site that is configured to be relatively displaced with respect to the first site and in which at least a portion of the second insertion hole is defined; and
by the second site being relatively displaced with respect to the first site, the main body is changed from the first state to the second state.

3. The blood bag system according to claim 2, wherein:
the main body includes a hinge portion configured to connect the first site and the second site;
by the second site being rotated about the hinge portion with respect to the first site, the main body is changed from the first state to the second state; and
the main body, when changing from the first state to the second state, forms a communication passage configured to allow the third tube to be detached from the second blocking portion and moved to an exterior of the main body.

4. The blood bag system according to claim 3, wherein:
the main body is formed in a plate-like shape; and
the second site is configured to be rotated about the hinge portion with respect to the first site in a thickness direction of the main body.

5. The blood bag system according to claim 3, wherein:
the main body is formed in a plate-like shape; and
the second site is configured to be rotated about the hinge portion with respect to the first site along a planar surface perpendicular to a thickness direction of the main body.

6. The blood bag system according to claim 4, wherein:
the second site includes an inner end forming a part of an edge portion that forms the second blocking portion; and
the main body includes:
a first separation line that is made up from a slit or a fragile portion configured to be broken, and that lies adjacent to the inner end of the second site at one end thereof, and adjacent to the hinge portion at another end thereof; and
a second separation line that is made up from a slit or a fragile portion configured to be broken, and that lies adjacent to the inner end of the second site at one end thereof.

7. The blood bag system according to claim 6, wherein:
the first separation line and the second separation line are both made up from the slits;
the one end of the first separation line is connected with the edge portion;
the one end of the second separation line is connected with the edge portion; and
another end of the second separation line is connected with an outer edge of the main body.

8. The blood bag system according to claim 3, wherein:
the first blocking portion and the second blocking portion respectively extend from the first opening portion and the second opening portion mutually in a same direction; and
the communication passage is formed in a direction that differs from a direction in which the second blocking portion extends.

9. The blood bag system according to claim 2, wherein:
the second site is configured to be separated away from the first site; and
by the second site being separated away from the first site, the main body is changed from the first state to the second state.

10. The blood bag system according to claim 9, wherein:
the second insertion hole includes a reopening portion connected with the second blocking portion at a position different from a position where the second blocking portion is connected with the second opening portion, the reopening portion being configured to allow the flow path of the third tube to be reopened;
the first site includes a projecting piece configured to restrict movement of the third tube from the second blocking portion into the reopening portion in the first state of the main body; and
by the restriction of the third tube by the projecting piece being released when the second site is separated away from the first site, movement of the third tube into the reopening portion is permitted.

11. The blood bag system according to claim 9, wherein, when the second site is separated away from the first site, the second site forms a communication passage configured to allow the third tube to be detached from the second blocking portion and moved to an exterior of the main body.

12. The blood bag system according to claim 2, wherein the main body includes, at a boundary between the first site and the second site, a locking mechanism configured to lock the first portion and the second portion in the first state.

13. A clamp mounted over two tubes, the clamp comprising:
a main body;
a first insertion hole that penetrates through the main body and in which one tube of the two tubes is arranged; and
a second insertion hole that penetrates through the main body and in which another tube of the two tubes is arranged, wherein
the first insertion hole includes a first opening portion configured to place a flow path of the one tube in an open state, and a first blocking portion connected with the first opening portion and configured to place the flow path of the one tube in a blocked state, and in the first blocking portion, a portion thereof except for a portion connected with the first opening portion is closed by the main body,
the second insertion hole includes a second opening portion configured to place a flow path of the another tube in an open state, and a second blocking portion connected with the second opening portion and configured to place the flow path of the another tube in a blocked state,
the main body is configured to be changed from a first state to a second state,
in the first state, the another tube, that has been moved into the second blocking portion from the second opening portion, is restrained by the second blocking portion, and
in the second state, the another tube is allowed to be moved from the second blocking portion to a position that is a position other than the second opening portion and in which the flow path of the another tube is reopened.

14. The clamp according to claim 13, further comprising:
a slit passing through the main body and extending from the second blocking portion through an outer periphery of the main body, wherein the slit separates a portion of the main body on a side of the second blocking portion.

15. The clamp according to claim 14, wherein the slit comprises a slit width, wherein the second blocking portion comprises a slot width, and wherein the slit width is sized less than the slot width.

16. The clamp according to claim 15, further comprising:
a hinge slit passing through the main body and extending from the second blocking portion to a hinge point inside the outer periphery of the main body on the side of the second blocking portion.

17. The clamp according to claim 16, wherein a deformation portion of the main body on the side of the second blocking portion between the slit and the hinge slit is configured to move between an undeformed state and a deformed state, wherein, in the undeformed state, the deformation portion of the main body is arranged in a plane of the main body, and wherein, in the deformed state, the deformation portion is arranged hinged about the hinge point out of the plane of the main body.

18. The clamp according to claim 17, wherein the deformation portion remains attached to the main body in the deformed state and in the undeformed state.

19. The clamp according to claim 17, wherein the slit includes at least one locking protrusion that prevents movement of the deformation portion out of the plane of the main body in a specific thickness direction.

20. A tube clamp assembly, comprising:
a first tube;
a second tube; and
a tube clamp engaged with the first tube and the second tube, the tube clamp, comprising:
a main body;
a first insertion hole that passes through a thickness of the main body, wherein the first insertion hole is configured to engage with the first tube, wherein the first insertion hole comprises:
a first opening portion comprising a first opening diameter; and
a first blocking portion comprising a first slot comprising a first slot width extending from the first opening diameter along a first slot length in the main body, and wherein the first opening diameter is greater than the first slot width; and
a second insertion hole that passes through the thickness of the main body, wherein the second insertion hole is configured to engage with the second tube, wherein the second insertion hole comprises:
a second opening portion comprising a second opening diameter; and
a second blocking portion comprising a second slot comprising a second slot width extending from the second opening diameter along a second slot length in the main body, and wherein the second opening diameter is greater than the second slot width;
a hinge slit extending from the second insertion hole to a hinge point of the main body; and
a separation slit passing through the main body and extending from the second insertion hole through an outer periphery of the main body, wherein the separation slit separates a portion of the main body on a side of the second insertion hole, and wherein a deformation portion of the main body on the side of the second insertion hole between the separation slit and the hinge slit is configured to move between an undeformed state and a deformed state, wherein, in the undeformed state, the deformation portion of the main body is arranged in a plane of the main body and the second tube is restrained by the second blocking portion, and wherein, in the deformed state, the deformation portion is arranged hinged about the hinge point out of the plane of the main body and the second tube is unrestrained by the second blocking portion.
